(19) United States
(12) Patent Application Publication
(10) Pub. No.: US 2026/0061079 A1
(43) Pub. Date: Mar. 5, 2026

(54) BBB interacting capsids

(71) Applicant: uniQure biopharma B.V., Amsterdam (NL)

(72) Inventors: - Anggakusuma, Amsterdam (NL); Elena Maria Santidrian Yebra-Pimentel, Amsterdam (NL)

(73) Assignee: uniQure biopharma B.V., Amsterdam (NL)

(21) Appl. No.: 19/307,083

(22) Filed: Aug. 22, 2025

(30) Foreign Application Priority Data
Aug. 22, 2024 (EP) 24195864.4
Oct. 23, 2024 (EP) 24208332.7
May 13, 2025 (EP) 25175939.5

(51) Int. Cl. A61K 48/00; A61K 38/17; C07K 14/005; C12N 15/86

(52) U.S. Cl. CPC A61K 48/0058; A61K 38/177; C07K 14/005; C12N 15/86; C12N 2750/14122; C12N 2750/14143

(57) ABSTRACT

The present invention relates to the fields of medicine, molecular biology, and gene therapy. In particular, the invention relates to novel recombinant adeno-associated virus capsids, uses thereof and methods of manufacturing the novel recombinant adeno-associated virus capsids.

### FIELD OF THE INVENTION

The present invention relates to the fields of medicine, molecular biology, and gene therapy. In particular, the invention relates to novel recombinant adeno-associated virus capsids, uses thereof and methods of manufacturing the novel recombinant adeno-associated virus capsids.

### REFERENCE TO AN ELECTRONIC SEQUENCE LISTING

The contents of the electronic sequence listing (sequencelisting.xml; Size: 330.914 bytes; and Date of Creation: Aug. 22, 2025) is herein incorporated by reference in its entirety.

### BACKGROUND OF THE INVENTION

Recombinant adeno-associated virus (rAAV) has emerged as a preferred platform for gene therapy. Yet, the development of novel rAAV-based therapeutic strategies has faced several hurdles. Specifically, developing novel rAAV capsids that can transduce cells in the central nervous system (CNS) upon systemic administration is one of the challenges in the field of rAAV-based gene therapy of CNS diseases. The inefficiency of blood-brain barrier (BBB) crossing limits the CNS application of first generation rAAV-based capsids due to the narrow therapeutic window when administered systemically. Traditional methods, such as intraparenchymal injection and intrathecal infusion, are invasive and may provide limited coverage. To address these challenges, research is being conducted on capsid engineering to develop novel rAAV capsids that can efficiently cross the BBB and achieve widespread CNS transduction via less invasive routes of administration, such as systemic delivery.

A major milestone in this field is the engineering of rAAV capsids via peptide insertion, which has led to the development of e.g. PHP.B rAAV capsid variants—the first to demonstrate efficacy in transducing the brain of C57BL/6 mice following systemic injection (Deverman et al., 2016, Nat Biotechnol., 34 (2): 204-209). However, these engineered rAAV capsid variants did not perform consistently across different mouse strains or in non-human primates (NHPs). Consequently, various research groups have employed different rAAV capsid engineering platforms to create engineered capsids with peptide insertions capable of crossing the BBB beyond rodent models and efficiently transducing the brain (Lopez-Gordo et al., Viruses 2024, 16(3), 442).

There remains, however, a need for further improved rAAV capsids that can cross the BBB and efficiently transduce cells in the CNS upon systemic administration. It is an object of the invention to provide engineered rAAV capsids with the capacity to cross the BBB.

### SUMMARY OF THE INVENTION

In a first aspect, there is provided a recombinant adeno-associated virus (rAAV) capsid comprising at least one binding moiety, wherein the at least one binding moiety confers to the rAAV capsid a phenotype of increased interaction with Transferrin receptor protein 1 (TfR1) as compared to a control rAAV capsid not comprising the binding moiety.

In one embodiment, the rAAV capsid presents at least one of the following phenotypes upon systemic administration: i) a phenotype of increased blood-brain barrier (BBB) crossing as compared to a control rAAV capsid not comprising the binding moiety; and/or ii) a phenotype of increased transduction of cells in the central nervous system (CNS) as compared to a control rAAV capsid not comprising the binding moiety.

In one embodiment, the binding moiety comprises an amino acid insert between two consecutive amino acids present within amino acids 580-595, preferably amino acids 585-590, more preferably amino acids 588 and 589 of an AAV9 capsid protein, or in an analogous position of a capsid protein of an AAV capsid serotype selected from the group comprising: AAV1, AAV2, AAV3A, AAV3B, AAV4, AAV5, AAV6, AAV7, AAV8, AAV10, AAV11, AAV12, AAV13 and AAVrh10, or modified versions thereof, preferably AAV6, or modified versions thereof.

In one embodiment, the binding moiety comprises at least 4, 5, 6, or 7 contiguous amino acids of an amino acid sequence X₁X₂X₃X₄X₅X₆X₇X₈ (SEQ ID NO: 295), wherein: X₁ is absent, or selected from: M, Y, R, L, T, A, I, P and F; X₂ is selected from: H, T, V, P, S, A, M, W, G, R, K, N and F; X₃ is selected from: R, L, M, K, Y, V, H, S, A, G, P and T; X₄ is absent, or selected from: L, F, Q, G and W; X₅ is selected from: L, H, S, P, R, T, A, Q and Y; X₆ is absent, or selected from: Q, L, V, P, I, R, S, T, W, N, Y, A and K; X₇ is selected from: T, Y, R, L, H, K, S, V, F, G and N, and; X₈ is absent, or selected from: Q, P, S, G, L and A; optionally wherein the amino acid at position 587 is mutated to D, and/or the amino acid at position 588 is mutated to G.

In one embodiment, the rAAV capsid comprises an expression cassette flanked by at least one AAV inverted terminal repeat (ITR), and the expression cassette comprises a nucleic acid molecule encoding at least one gene product.

In a second aspect, there is provided a polynucleotide encoding an rAAV capsid as described herein.

In a third aspect, there is provided a host cell comprising a polynucleotide for expression of the rAAV capsid as described herein.

In a fourth aspect, there is provided a composition comprising the rAAV capsid as described herein, preferably wherein the composition is a pharmaceutical composition further comprising at least one pharmaceutically acceptable carrier.

In a fifth aspect there is provided an rAAV capsid as described herein, or a composition as described herein for use as a medicament, preferably for use in the treatment of a condition of the central nervous system.

In a sixth aspect, there is provided an rAAV capsid as described herein, or a composition as described herein, for use as a medicament in gene therapy.

In one embodiment, there is provided a method of gene therapy comprising the step of administering an effective amount of an rAAV capsid as described herein, or a composition comprising the rAAV capsid as described herein, to a subject in need of gene therapy.

In a seventh aspect, there is provided a method of manufacturing an rAAV capsid as described herein, the method comprising the steps of: I) culturing a host cell comprising: i) an expression vector for expression of a rAAV capsid as described herein, and; ii) an expression vector encoding an expression cassette flanked by at least one one AAV inverted terminal repeat (ITR), comprising a nucleic acid molecule encoding at least one gene product; said host cell preferably further comprising a nucleotide sequence encoding a parvoviral replication (Rep) protein under conditions such that the rAAV capsid is produced; and, II) recovery of the rAAV capsid, preferably wherein recovery of the rAAV capsid comprises at least one of: affinity-purification of the rAAV capsid using an immobilized anti-AAV antibody, preferably a single chain camelid antibody or a fragment thereof, or filtration using a filter having a nominal pore size of 30-70 nm.

### DESCRIPTION OF THE INVENTION

The present invention surprisingly provides multiple rAAV capsid variants that interact with human Transferrin Receptor (hTfR), enabling BBB crossing of the capsids.

### Recombinant adeno-associated virus (rAAV) capsids

In a first aspect, there is provided a recombinant adeno-associated virus (rAAV) capsid comprising at least one binding moiety, wherein the at least one binding moiety confers to the rAAV capsid a phenotype of increased interaction with Transferrin Receptor protein 1 (TfR1) as compared to a control rAAV capsid not comprising the binding moiety.

The term “capsid” as used herein may interchangeably be denominated by the terms “viral vector”, “virion”, “vector virion”, and/or “particle”. The term “capsid” as used herein may refer to a collection of capsid proteins that together assemble into an AAV or rAAV capsid, and may as well refer to the combination of an AAV or rAAV capsid and any cargo that is contained within the capsid. Exemplary cargo includes, but is not limited to an expression vector.

The term “recombinant” in relation to the capsids as described herein is used to indicate that recombinant AAV capsids comprise mutations that that were intentionally introduced, distinguishing them from wild type AAV capsids having the same serotype. This means that the amino acid sequence of the rAAV capsid as described herein is non-naturally occurring. As used herein, the term “non-naturally occurring” when used in reference to an rAAV capsid means that the rAAV capsid comprises at least one mutation that is not normally found in naturally occurring AAV capsids, including wild-type AAV capsids.

The term “binding moiety” as used herein refers to a peptide, a polypeptide or oligopeptide having the capacity to specifically bind to a certain tertiary or quaternary protein structure or epitope. In some embodiments, a binding moiety as referred to herein may also consist of or comprise carbohydrate chains having the capacity to specifically bind to a certain tertiary or quaternary protein structure or epitope.

The terms “interaction” and “interacting” as used herein may be used interchangeably with terms including, but not limited to: “association, associating, connection, connecting, binding, engaging”. The term interaction as used herein indicates that two entities specifically bind to each other, but that the binding is of a transient nature, preferably wherein the binding event is of a transient nature. This transient nature of the binding event allows the rAAV capsids as described herein to cross the BBB, followed by release into the CNS.

The term “control rAAV capsid not comprising the binding moiety”, “control rAAV capsid not capable of interacting with TfR1” and “control rAAV capsid” as used herein may be used interchangeably and refer to any AAV or rAAV capsid of any serotype. The control rAAV capsid should not comprise a binding moiety that interacts with TfR1, and the rAAV serotype should not intrinsically interact with TfR1 to a higher degree as compared to wild type AAV capsids of the different serotypes, including, but not limited to: AAV1, AAV2, AAV3A, AAV3B, AAV4, AAV5, AAV6, AAV7, AAV8, AAV9, AAV10, AAV11, AAV12, AAV13 and AAVrh10, preferably AAV9. The degree to which a control capsid interacts with TfR1, including murine, macaque and human TfR1, may be determined using methods as described herein.

Where herein reference is made to “Transferrin receptor protein 1 (TfR1)”, it is understood to refer to human Transferrin receptor protein 1″ (hTfR1). Therefore, in one embodiment there is provided a recombinant adeno-associated virus (rAAV) capsid comprising a binding moiety, wherein the binding moiety confers to the rAAV capsid a phenotype of increased interaction with human Transferrin receptor protein 1 (TfR1) as compared to a control capsid not comprising the binding moiety.

The term “Transferrin receptor protein 1 (TfR1)” may also be referred to herein as “Transferrin receptor” and/or its abbreviations: “TfR”, “TR”, “TfR1”, and/or “Trfr” as well as its cluster of differentiation (CD) antigen name “CD71”. Human TfR1 is herein understood to comprise, consist of, or to be comprised within the amino acid denominated by UniProt® Database Accession No. P02796 and the nucleotide sequence denominated by HGNC ID: 11763 (TFRC). TfR1 is a transmembrane glycoprotein that is composed of two monomers that are joined by two disulfide bonds and is highly expressed on endothelial cells of the Blood-Brain Barrier (BBB). Without being bound by theory, it is believed that capsids as described herein when present in the blood can associate with TfR1 expressed on cells present on the luminal side of the BBB via binding moieties as described herein. Upon association with TfR1 the capsids can leave the blood circulation and enter the cerebrospinal fluid (CSF) through a transcellular or possibly paracellular mechanism. Upon dissociation from TfR1, the capsids of the invention are released into the CSF, enabling the capsids to transduce target cells of the CNS, e.g. astrocytes, microglia and neurons.

In one embodiment, the phenotype of increased interaction with TfR1 is determined by incubating an rAAV capsid as described herein comprising a reporter gene, with TfR1-expressing cells, preferably TfR1-expressing Human Embryonic Kidney (HEK) 293T cells, and determining the number of cells that are positive for the reporter gene, and preferably comparing the number of cells that are positive for the reporter gene upon incubation with an rAAV capsid as described herein with the number of cells that are positive for the reporter gene when incubated with a control rAAV capsid not capable of interacting with TfR1, preferably under otherwise identical conditions.

In one embodiment, the number of cells that are positive for the reporter gene is at least 2, 3, 4, 5, 10, 15, 20, 40, 50, 60, 75 or 100 fold higher when incubated with an rAAV capsid as described herein as compared to a control rAAV capsid not capable of interacting with TfR1.

Exemplary methods for determining interaction of rAAV capsids as described herein with TfR1 include, but are not limited to: polymerase-chain-reaction-based methods, protein expression analysis, deep sequencing, electron microscopy and fluorescence microscopy.

In one embodiment, the rAAV capsid presents at least one of the following phenotypes upon systemic administration: i) a phenotype of increased blood-brain barrier (BBB) crossing as compared to a control rAAV capsid not comprising the binding moiety; and/or ii) a phenotype of increased transduction of cells in the central nervous system (CNS) as compared to a control rAAV capsid not comprising the binding moiety.

Where herein reference is made to “blood-brain barrier (BBB) crossing” it is intended to refer to the crossing of the BBB from the lumen of the blood vessel towards the CNS.

In one embodiment, the phenotype of increased BBB crossing is determined in vivo, by administering an rAAV capsid comprising a binding moiety as described herein to a non-human animal, preferably a mammal, and determining whether rAAV capsids reach the CNS, and preferably comparing the number of rAAV capsids that are present in the CNS to the number of control rAAV capsids that are present in the CNS, preferably under otherwise identical conditions. In a preferred embodiment, the phenotype of BBB crossing is determined upon systemic administration of the rAAV capsids as described herein to a non-human animal.

Exemplary methods for determining the presence of rAAV capsids in the CNS include, but are not limited to: polymerase-chain-reaction-based methods, protein expression analysis, deep sequencing, electron microscopy and fluorescence microscopy.

In one embodiment, the phenotype of BBB crossing is determined 1, 4, 8, 24 or 48 hours post-administration. In one embodiment, the number of capsids that is present in the CNS is at least 2, 3, 4, 5, 10, 15, 20, 40, 50, 60, 75 or 100-fold higher upon administration of an rAAV capsid as described herein, as compared to a control rAAV capsid. In one embodiment, the non-human animal is a mouse or other laboratory research animal, such as, but not limited to, a rabbit, a rat, a dog or a non-human primate.

In one embodiment, the phenotype of increased BBB crossing is determined using an in vitro method as provided in the examples.

In one embodiment, the phenotype of increased transduction of cells in the CNS is determined by administering an rAAV capsid as described herein to a non-human animal, preferably a mammal, and determining the transduction levels of cells in the CNS, and preferably comparing the transduction levels obtained using an rAAV capsid as described herein with the transduction levels obtained using a control rAAV capsid, preferably under otherwise identical conditions. In a preferred embodiment the phenotype of increased transduction of cells in the CNS is determined upon systemic administration of the rAAV capsids as described herein to a non-human animal.

In one embodiment, the phenotype of increased transduction of cells in the CNS is determined 2, 3, 4, 5 or 6 weeks post-administration. In one embodiment, the transduction level of cells in the CNS is at least 2, 3, 4, 5, 10, 15, 20, 40, 50, 60, 75 or 100-fold higher upon administration of an rAAV capsid as described herein, as compared to a control rAAV capsid. In one embodiment, the non-human animal is a mouse or other laboratory research animal, such as, but not limited to, a rabbit, a rat, a dog or a non-human primate.

Exemplary methods for determining transduction of cells in the CNS include, but are not limited to: polymerase-chain-reaction-based methods, protein expression analysis, deep sequencing, electron microscopy and fluorescence microscopy.

In one embodiment, the phenotype of increased transduction of cells in the CNS is determined using an in vitro method as provided in the examples.

In one embodiment, at least part of the binding moiety comprised in the rAAV capsid is inserted between two consecutive amino acids of a capsid protein.

The term “at least part of” as used herein means to indicate that at least 1 amino acid, but possibly more, e.g. 2, 3, 4, 5, 6, 7 or more amino acids of the binding moiety are additional to the amino acid sequence of a wild type AAV capsid protein. This means that the amino acid sequence of a capsid protein constituting the rAAV capsid as described herein is at least 1 amino acid, possibly more, e.g. 2, 3, 4, 5, 6, 7, or more amino acids longer as compared to the amino acid sequence of a corresponding wild type AAV capsid protein.

The term “inserted between” as used herein refers to an insertion or addition of additional amino acids into the polypeptide constituting an rAAV capsid protein.

The term “consecutive amino acids” refers to the situation wherein for example a first amino acid of a polypeptide is followed by a second amino acid, or a fifth amino acid is followed by a sixth amino acid. In one embodiment, at least part of the binding moiety as described herein is added between two amino acids that are adjacent in at least one polypeptide that is part of an AAV capsid protein.

AAV capsids are assembled from viral proteins VP1, VP2 and VP3. In one embodiment, the binding moiety comprised in the rAAV capsid is inserted between two consecutive amino acids of at least one of VP1, VP2 and VP3. In a preferred embodiment, the binding moiety is inserted between two consecutive amino acids of VP1, VP2 and VP3. Together, multiple VP1, VP2 and VP3 monomers assemble into an AAV capsid.

In one embodiment, the rAAV capsid as described herein comprises at least two binding moieties, wherein a first binding moiety is present in a first viral protein, and a second binding moiety is present in a second viral protein, wherein the first and second viral protein are not the same.

In another embodiment, the rAAV capsid as described herein comprises at least two binding moieties, wherein a first binding moiety is present in a first viral protein, and a second binding moiety is present in the same viral protein.

In a preferred embodiment, the first binding moiety interacts with TfR1, and the second binding moiety interacts with a different protein.

In a preferred embodiment, the first binding moiety interacts with TfR1, and the second binding moiety interacts with a protein that is expressed on the surface of human brain microvascular endothelial cells (BMEC).

Exemplary proteins that are expressed on the surface of human brain microvascular endothelial cells (BMEC) include, but are not limited to: Carbonic Anhydrase 4 (CA4, UniProt® Database Accession No. P22748), Cell cycle control protein 50A (UniProt® Database Accession No. Q9NV96), alkaline phosphatase-tissue-nonspecific isozyme (TNS-ALP, UniProt® Database Accession No. P05186), Low-density lipoprotein receptor-related protein 6 (LRP-6, UniProt® Database Accession No. 075581), and CD59 glycoprotein (CD59, UniProt® Database Accession No. P13987).

In a preferred embodiment, the at least one binding moiety is external to the rAAV capsid. The terms “external to the rAAV capsid” and “on the surface of the rAAV capsid” as used herein may be used interchangeably and refer to the amino acids forming secondary and tertiary structures facing outside of the rAAV capsid, as opposed to the amino acids forming secondary and tertiary structures facing the inside of the rAAV capsid. It may thus be interpreted as such, that the amino acids that are part of the binding moiety are part of a secondary and tertiary structure of the rAAV capsid facing outside of the rAAV capsid.

In one embodiment the binding moiety is comprised in a variable region (VR) of a surface loop of the rAAV capsid, preferably in at least one of the VR-I, VR-II, VR-III, VR-IV, VR-V, VR-VI, VR-VII, VR-VIII and/or VR-IX, preferably in the VR-IV and/or VR-VIII, more preferably in the VR-VIII.

AAV viral proteins have nine variable regions (VRs), which are oriented external to the AAV capsid. Individually, the variable regions shape the exterior of the AAV capsid. In a preferred embodiment, the binding moiety is comprised in the VR-VIII.

In one embodiment, the binding moiety comprised in the rAAV capsid comprises an amino acid insert between two consecutive amino acids present within amino acids 580-595, preferably amino acids 585-590, more preferably amino acids 588 and 589 of an AAV9 capsid protein, or in an analogous position of a capsid protein of an AAV capsid serotype selected from the group comprising: AAV1, AAV2, AAV3A, AAV3B, AAV4, AAV5, AAV6, AAV7, AAV8, AAV10, AAV11, AAV12, AAV13 and AAVrh10, or modified versions thereof, preferably AAV6 or modified versions thereof.

The skilled person will be aware that where herein reference is made to numbered amino acids, the amino acid numbered 1 is the amino-terminal amino acid of the capsid protein, from there, the amino acids count up or upwards sequentially and monotonically until the carboxy-terminal amino acid of the capsid protein. In one embodiment, the numbered amino acids referred to herein do not include the amino acids that constitute the insert itself, but refer to amino acids that were present in the (recombinant) AAV capsid protein prior to addition of the insert.

In one embodiment, the sequences coding for the VP1, VP2, and VP3 capsid proteins for use in the context of the present invention may be taken from any of the known 42 serotypes, more preferably from AAV1, AAV2, AAV3, AAV4, AAV5, AAV6, AAV7, AAV8. AAV9, AAV10, AAV11, AAV12, AAV13, AAVrh10, or AAVrh74, or from newly developed rAAV capsids obtained by e.g. capsid shuffling techniques.

In a further embodiment, modified rAAV capsids, or “modified versions” are used. Modified rAAV capsids have at least 70%, at least 75%, at least 80%, at least 85%, at least 90%, at least 95%, or more nucleotide and/or amino acid sequence identity, e.g., a sequence having 75-99% nucleotide sequence identity, to an AAV1, AAV2, AAV3, AAV4, AAV5, AAV6, AAV7, AAV8, AAV9, AAV10, AAV11, AAV12, AAV13, AAVrh10, or AAVrh74 capsid.

In a preferred embodiment, the rAAV capsid is an rAAV9 capsid, preferably comprising an amino acid sequence selected from SEQ ID NO: 298, 302, or 303. More preferably, the AAV9 capsid consists of an amino acid sequence selected from SEQ ID NO: 298, 302, or 303.

Where herein reference is made to “an analogous position of a capsid protein”, it is intended to refer to a position in a capsid protein of a serotype other than AAV9, that corresponds to a similar position as the position indicated in the AAV9 capsid protein. Such similarity may occur for instance due to a similar position of amino acids within a certain VR region. Analogous positions of a capsid protein may be determined using software, including protein or sequence-BLAST.

Without being bound to theory, AAV-serotypes that have similar capsid protein amino acid sequences, and comprise at least one binding moiety as described herein in analogous positions are expected to have similar functionality. Exemplary AAV-serotypes with similar capsid protein amino acid sequences include, but are not limited to AAV9 and AAV6.

In one embodiment, the amino acid insert comprised in the binding moiety consists of 2, 3,4, 5, 6, 7, 8, 9, 10, 11, 12, 13, 14, 15 or more amino acids, preferably 5-9 amino acids, more preferably 7 amino acids.

In one embodiment, the rAAV capsid comprises a binding moiety comprising at least 4, 5, 6, or 7 contiguous amino acids of an amino acid sequence X₁X₂X₃X₄X₅X₆X₇X₈ (SEQ ID NO: 295), wherein: X₁ is absent, or selected from: M, Y, R, L, T, A, I, P and F; X₂ is selected from: H, T, V, P, S, A, M, W, G, R, K, N and F; X₃ is selected from: R, L, M, K, Y, V, H, S, A, G, P and T; X₄ is absent, or selected from: L, F, Q, G and W; X₅ is selected from: L, H, S, P, R, T, A, Q and Y; X₆ is absent, or selected from: Q, L, V, P, I, R, S, T, W, N, Y, A and K; X₇ is selected from: T, Y, R, L, H, K, S, V, F, G and N, and; X₈ is absent, or selected from: Q, P, S, G, L and A; optionally wherein the amino acid at position 587 is mutated to D, and/or the amino acid at position 588 is mutated to G. In a preferred embodiment, X₁ is inserted after amino acid 588 and X₈ is followed by amino acid 589 of an AAV9 capsid protein, or in an analogous position of a capsid protein of an AAV capsid serotype selected from the group comprising: AAV1, AAV2, AAV3A, AAV3B, AAV4, AAV5, AAV6, AAV7, AAV8, AAV10, AAV11, AAV12, AAV13 and AAVrh10, or modified versions thereof, preferably AAV6 or modified versions thereof.

In one embodiment, the rAAV capsid protein comprises or consists of SEQ ID NO:298, wherein X₁ is inserted after amino acid 588 and X₈ is followed by amino acid 589.

In a preferred embodiment, the rAAV capsid protein comprises or consists of SEQ ID NO:298, 302 or 303, wherein X₁ is inserted after amino acid 588 and X₈ is followed by amino acid 589. In a preferred embodiment, the rAAV capsid protein comprises or consists of SEQ ID NO: 301, or a modified version thereof. In one embodiment, the modified AAV capsid protein has at least 70%, or at least 75%, or at least 80%, or at least 85%, preferably at least 90%, more preferably at least 95%, or more amino acid sequence identity with SEQ ID NO: 301.

In one embodiment, the rAAV capsid protein comprises or consists of SEQ ID NO:298, wherein X₁ is inserted after amino acid 588 and X₇ is followed by amino acid 589.

In a preferred embodiment, the rAAV capsid protein comprises or consists of SEQ ID NO:298, 302 or 303, wherein X₁ is inserted after amino acid 588 and X₇ is followed by amino acid 589. In a preferred embodiment, the rAAV capsid protein comprises or consists of SEQ ID NO: 301, or a modified version thereof. In one embodiment, the modified AAV capsid protein has at least 70%, or at least 75%, or at least 80%, or at least 85%, preferably at least 90%, more preferably at least 95%, or more amino acid sequence identity with SEQ ID NO: 301.

In a preferred embodiment, the rAAV capsid protein comprises or consists of SEQ ID NO: 301, 307 or 308, or a modified version thereof. In one embodiment, the modified AAV capsid protein has at least 70%, or at least 75%, or at least 80%, or at least 85%, preferably at least 90%, more preferably at least 95%, or more amino acid sequence identity with SEQ ID NO: 301, 307 or 308.

In a preferred embodiment, the rAAV capsid comprises a binding moiety comprising a consensus sequence AQX₁X₂X₃X₄X₅X₆X₇X₈ (SEQ ID NO: 296), wherein X₁ is absent, or selected from: M, Y, R, L, T, A, I and F; X₂ is selected from: H, T, V, P, S, A, M, W, G, R, K, N and F; X₃ is selected from: R, L, M, K, Y, V, H, S, A, G and T; X₄ is absent, or selected from: L, F, Q and G; X₅ is selected from: L, H, S, P, R, T, A, Q and Y; X₆ is absent, or selected from: Q, L, V, P, I, R, S, T, W, N, Y, A and K; X₇ is selected from: T, Y, R, L, H, K, S, V, F, G and N, and; X₈ is absent, or selected from: Q, P, S, G, L and A; or, wherein the binding moiety comprises the amino acid sequence DGPVPWRVR (SEQ ID NO: 23).

In a preferred embodiment, the rAAV capsid comprises a binding moiety comprising a consensus sequence AQX₁X₂ X₃X₄X₅X₆X₇ (SEQ ID NO: 299), wherein X₁ is selected from M, Y, L, and R; X₂ is selected from H and T; X₃ is selected from R and L; X₄ is selected from L and S; X₅ is selected from Q, H, T and V; X₆ is selected from T, L, V and Y, and; X₇ is selected from Q, T, G and P.

In a further preferred embodiment, the rAAV capsid comprises a binding moiety comprising a consensus sequence AQX₁X₂RLX₃X₄X₅ (SEQ ID NO: 300), wherein X₁ is selected from M, Y and L; X₂ is selected from H and T; X₃ is selected from Q, H and T; X₄ is selected from T, L and V, and; X₅ is selected from Q, T and G.

In a further embodiment, the rAAV capsid comprises a binding moiety, wherein the binding moiety comprises or consists of an amino acid sequence selected from SEQ ID NO: 1-147. In a preferred embodiment the amino acid sequence is selected from SEQ ID NO: 1-26, more preferably selected from SEQ ID NO: 1-3 and 16.

In another embodiment, the rAAV capsid comprises a binding moiety, wherein the binding moiety comprises or consists of a nucleic acid sequence selected from SEQ ID NO: 148-294. In a preferred embodiment the nucleic acid sequence is selected from SEQ ID NO: 148-173, more preferably the nucleic acid sequence is selected from SEQ ID NO: 148-150 and 163.

In a preferred embodiment, the rAAV capsid protein comprises or consists of SEQ ID NO: 304, 305 or 306, or a modified version thereof. In one embodiment, the modified AAV capsid protein has at least 70%, or at least 75%, or at least 80%, or at least 85%, preferably at least 90%, more preferably at least 95%, or more amino acid sequence identity with SEQ ID NO: 304, 305 or 306. In further preferred embodiments, the rAAV capsid protein consists of SEQ ID NO: 304, 305 or 306, preferably the rAAV capsid protein consists of SEQ ID NO: 304.

### Expression Cassette

In one embodiment, the rAAV capsid comprises an expression cassette flanked by at least one AAV inverted terminal repeat (ITR), wherein the expression cassette comprises a nucleic acid molecule encoding at least one gene product.

Wild type AAV capsids carry a single stranded DNA genome, which consists of two open reading frames carrying the replicase genes and the capsid genes, Rep and Cap, and is flanked by two inverted terminal repeats (ITRs). In recombinant AAV capsids as described herein, the single stranded DNA genome is replaced with an expression cassette.

The term “expression cassette” as used herein describes the functional unit capable of affecting expression of a nucleic acid molecule encoding a gene product. A “nucleic acid molecule encoding a gene product” as referred to herein can be defined as a nucleic acid molecule which comprises a nucleotide sequence encoding at least one gene product.

The nucleic acid molecule encoding a gene product is operably linked to the appropriate expression control sequences, which at least comprises a suitable transcription regulatory sequence and optionally, 3′ transcription termination signals. Additional factors necessary or helpful in affecting expression may also be present, such as expression enhancer elements.

In some embodiments, the nucleic acid molecule encoding a gene product is operably linked to a polyadenylation or polyA or pA signal. The polyadenylation signal aids in the nuclear export of RNA and RNA translation and promotes RNA transcript longevity. In a preferred embodiment, the polyA is located at the 3′ end of the expression cassette upstream of the 3′ ITR.

As used herein, the term “transcription regulatory sequence” or “promoter” refers to a nucleic acid fragment that functions to control the transcription of one or more coding nucleotide sequences, and is located upstream with respect to the direction of transcription of the transcription initiation site of the coding nucleotide sequence. The “transcription regulatory sequence” or “promoter” is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the transcription regulatory sequence or promoter. A “constitutive” transcription regulatory sequence or promoter is a transcription regulatory sequence or promoter that is active under most physiological and developmental conditions. An “inducible” transcription regulatory sequence or promoter is a transcription regulatory sequence or promoter that is physiologically or developmentally regulated, e.g. by the application of a chemical inducer or biological entity.

In one embodiment, the expression cassette is flanked by at least one inverted terminal repeat (ITR).

The term “flanked” with respect to a sequence that is flanked by another element(s) herein indicates the presence of one or more of the flanking elements upstream and/or downstream, i.e., 5′ and/or 3′, relative to the sequence. The term “flanked” is not intended to indicate that the sequences are necessarily contiguous. For example, there may be intervening sequences between the nucleic acid encoding the gene product and a flanking element. A sequence that is “flanked” by two other elements (e.g. ITRs), indicates that one element is located 5′to the sequence and the other is located 3′ to the sequence; however, there may be intervening sequences there between.

In one embodiment, the expression cassette is flanked by two AAV ITR nucleotide sequences, preferably wherein the expression cassette is located in between the two AAV ITR nucleotide sequences, more preferably AAV2 serotype ITR nucleotide sequences or ITR nucleotide sequences which have at least 70%, at least 75%, at least 80%, at least 85%, at least 90%, at least 95%, or more sequence identity with an AAV2 serotype ITR. In another embodiment, the expression cassette is flanked by one ITR engineered with two D regions, wherein preferably the expression cassette is located on either side of the engineered ITR.

In one embodiment, the nucleic acid molecule comprising an expression cassette and at least one ITR as described herein is 5,000 nucleotides (nt) or less in length. The skilled person is aware that the maximum AAV packaging limit is understood to be 5.5 kilobasepairs (kbp). However, in further embodiments, wherein oversized nucleic acid molecules are used, e.g. nucleic acid molecules of more than 5,000 nt in length, or even more than the maximum AAV packaging limit of 5.5 kbp may also still allow incorporation of the nucleic acid molecule in rAAV capsids as described herein and such embodiments are therefore not excluded.

### Promoter

In one embodiment, the expression cassette as described herein comprises a transcription regulatory sequence and/or promoter. In a further embodiment, the transcription regulatory sequence and/or promoter is mammalian cell compatible, preferably the transcription regulatory sequence and/or promoter is operably linked to the nucleotide sequence encoding for at least one product. Many mammalian cell compatible promoters are known in the art (Sambrook and Russell, Molecular Cloning: A laboratory Manual. 3rd Edition, Cold Spring Harbor Laboratory Press, New York, (2001) 1.31-31.42 and 31.59-31.61.).

In one embodiment, the promoter is a constitutive or ubiquitous promoter that is broadly expressed in many cell-types. Exemplary constitutive or ubiquitous promoters include, but are not limited to the Cytomegalovirus (CMV) promoter, the CMV early enhancer/chicken β-actin (CAG) promoter, the chicken β-actin (CBA) promoter and the phosphoglycerate kinase-1 (PGK) promoter.

In a further embodiment, the promoter is an inducible, tissue-specific, cell-type-specific, or cell cycle-specific promoter.

In a preferred embodiment, the promoter is a neuron-specific promoter. Exemplary neuron-specific promoters include, but are not limited to: a neuron-specific enolase (NSE) promoter, platelet-derived growth factor (PDGF) promoter, platelet-derived growth factor B-chain (PDGF-(3) promoter, synapsin or synapsin-1 (Syn or Syn-1) promoter, methyl-CpG binding protein 2 (MeCP2) promoter, Ca+/calmodulin-dependent protein kinase II (CaMKII) promoter, metabotropic glutamate receptor 2 (mGluR2) promoter, neurofilament light (NFL) or heavy (NFH) promoter, β-globin minigene nβ2 promoter, preproenkephalin (PPE) promoter, enkephalin (Enk) promoter and excitatory amino acid transporter 2 (EAAT2) promoter.

In a further embodiment, the promoter is an astrocyte-specific promoter. Exemplary astrocyte-specific promoters include, but are not limited to: glial fibrillary acidic protein (GFAP) and EAAT2 promoters.

In a further embodiment, the promoter is an oligodendrocyte-specific promoter. An exemplary oligodendrocyte-specific promoter includes, but is not limited to the myelin basic protein (MBP) promoter.

In a particularly preferred embodiment, the promoter for expression of a gene product in the central nervous system is the CBh promoter (Gray et al., Hum. Gene Ther. 2011, 22:1143-1153).

### Gene Product

As discussed above, in some embodiments, the rAAV capsid as described herein comprises an expression cassette flanked by at least one ITR, wherein the expression cassette comprises a nucleic acid molecule encoding at least one gene product.

The term “gene product” as used herein refers to a nucleic acid molecule encoding at least one RNA molecule and/or encoding at least one polypeptide, or a combination thereof. In a preferred embodiment, the nucleotide sequence encodes a gene product for expression in the mammalian cell and/or encodes a gene product for silencing expression of a gene of interest in a mammalian cell. A particularly preferred mammalian cell in which the “gene product” is to be expressed, is a human cell.

In one embodiment, an expression cassette as described herein may comprise a nucleic acid molecule encoding at least two different gene products, including, but not limited to at least two of the same gene product, or at least two different gene products. Such gene products include, but are not limited to, RNAi agents and polypeptides, as well as combinations thereof.

The term “RNAi agents” as used herein refers to RNA molecules that are capable of RNA interference such as, e.g. a shRNA (short hairpin RNA) a siRNA (short interfering RNA), or a miRNA (microRNA). The term “siRNA” refers to a small interfering RNA that is a short-length double-stranded RNA that can inhibit gene expression in a post-transcriptional matter and are usually not toxic in mammalian cells (Elbashir et al., 2001, Nature 411:494-98; Caplen et al., 2001, Proc. Natl. Acad. Sci. USA 98:9742-47). Similarly to siRNAs, miRNAs also inhibit gene expression in a post-transcriptional manner. Although the gene silencing effects of siRNAs and miRNAs are distinct, the distinction has been obscured because they are associated with common enzymes (e.g., Dicer and RISC) and their functions overlap with each other to a certain extent. The major difference between siRNAs and miRNAs is that the former inhibit the expression of one specific target mRNA while the latter regulate the expression of multiple mRNAs (Lam et al., 2015, Mol Ther Nucleic Acids. 2015 Sep. 15; 4(9):e252).

In one embodiment, the gene product as described herein is a therapeutic gene product. The term “therapeutic gene product” as used herein can refer to a polypeptide, or an (non-coding) RNA molecule (si/sh/miRNA), or other gene product that, when expressed in a target cell, provides a desired therapeutic effect. A desired therapeutic effect may be the ablation of an undesired activity, the complementation of a genetic defect, the silencing of genes that cause disease, the restoration of a deficiency in an enzymatic activity or any other disease-modifying effect. Examples of therapeutic polypeptide gene products include, but are not limited to growth factors, factors that form part of the coagulation cascade, enzymes, lipoproteins, cytokines, neurotrophic factors, hormones and therapeutic immunoglobulins and variants thereof.

In one embodiment, there is provided an rAAV capsid as described herein, comprising an expression cassette flanked by at least one ITR, wherein the expression cassette comprises a nucleic acid molecule encoding at least one gene product having an effect on an CNS disease.

In one embodiment, there is provided an rAAV capsid as described herein, comprising an expression cassette flanked by at least one ITR, wherein the expression cassette comprises a nucleic acid molecule encoding an RNA molecule and/or polypeptide having an effect on an CNS disease. In a preferred embodiment, an RNA molecule and/or polypeptide as described herein slows down the progression of a CNS disease. In a further embodiment, an RNA molecule and/or polypeptide as described herein, slows down the deterioration of the condition of a subject suffering from a CNS disease. In one embodiment, an RNA molecule and/or polypeptide as described herein eliminates one or more symptoms of a CNS disease.

In one embodiment, the expression cassette may comprise a nucleotide sequence encoding a polypeptide that serves as a selection marker protein to assess cell transformation and expression. Suitable marker proteins for this purpose include, but are not limited to: the fluorescent protein GFP, and the selectable marker genes HSV thymidine kinase (for selection on HAT medium), bacterial hygromycin B phosphotransferase (for selection on hygromycin B), Tn5 aminoglycoside phosphotransferase (for selection on G418), and dihydrofolate reductase (DHFR) (for selection on methotrexate), CD20, the low affinity nerve growth factor gene.

### Polynucleotides

In a second aspect, there is provided a polynucleotide encoding an rAAV capsid as described herein.

The term “polynucleotide” as used herein refers to a nucleic acid molecule comprising a multitude of nucleotides. The terms “polynucleotide” may be used interchangeably with the term “nucleic acid molecule”.

### Host Cell

In a third aspect, there is provided a host cell comprising a polynucleotide for expression of the rAAV capsid as described herein.

In a preferred embodiment, the polynucleotide is an expression vector for expression of an rAAV capsid as described herein.

In one embodiment, the expression vector for expression of the rAAV capsid as described herein is an insect cell-compatible vector or a mammalian cell-compatible vector. A “mammalian cell-compatible vector” is understood to be a nucleic acid molecule capable of productive transformation or transfection of a mammalian cell or cell line. Mammalian cell-compatible vectors are well-known in the art. An “insect cell-compatible vector” is understood to be a nucleic acid molecule capable of productive transformation or transfection of an insect or insect cell. Exemplary insect cell-compatible vectors include plasmids, linear nucleic acid molecules, and recombinant viruses, such as baculoviruses. Any vector can be employed as long as it is insect cell- or mammalian cell-compatible. The mammalian or insect cell-compatible vector may integrate into the cell's genome but the presence of the vector in the cell need not be permanent and transient episomal vectors are also included. The vectors can be introduced by any means known, for example by chemical treatment of the cells, electroporation, infection or transduction. The expression vector will be introduced into a suitable host cell and be able to effect expression of the coding sequence in an in vitro cell culture of the host cell. A preferred expression vector will be suitable for expression of viral proteins and/or nucleic acids, particularly recombinant parvoviral proteins and/or nucleic acids, such as baculoviral vectors for expression of parvoviral proteins and/or nucleic acids in insect cells.

A host cell as described herein may be any cell that is suitable for the production of heterologous proteins. In one embodiment, the host cell as described herein is an insect cell, more preferably, an insect cell that allows for replication of baculoviral vectors and can be maintained in culture. In a preferred embodiment, the insect cell allows for expression of at least one baculoviral expression vector such that recombinant parvoviral vectors, including rAAV vectors are produced. Exemplary host cells include, but are not limited to cells derived from: Spodoptera frugiperda, Drosophila cell lines, or mosquito cell lines, including Aedes albopictus-derived cell lines. In a preferred embodiment, the host cells are derived from insect species which are susceptible to baculovirus infection, including, but not limited to: S2 (CRL-1963, ATCC), Se301, SelZD2109, SeUCR1, Sf9, Sf900+, Sf21, BTI-TN-5B1-4, MG-1, Tn368, HzAm1, Ha2302, Hz2E5, High Five (Invitrogen, CA, USA) and expresSF+® (U.S. Pat. No. 6,103,526; Protein Sciences Corp., CT, USA). In a preferred embodiment, a host cell as described herein is an insect cell for production of rAAV capsids, preferably an expesSF+® cell.

In one embodiment, the host cell as described herein is a mammalian cell, more preferably, a mammalian cell that allows for expression and/or replication of mammalian cell-compatible expression vectors and can be maintained in culture. Exemplary mammalian cell lines for producing AAV vectors include, but are not limited to: A549, WEHI, 3T3, 10T1/2, BHK, MDCK, COS 1, COS 7, BSC 1, BSC 40, BMT 10, VERO, WI38, HeLa, a HEK 293 cell, Saos, C2C12, L cells, HT1080, HepG2 and primary fibroblast, hepatocyte and myoblast cells derived from mammals including human, monkey, mouse, rat, rabbit, and hamster. The selection of the mammalian species providing the cells is not a limitation of this disclosure; nor is the type of mammalian cell, i.e., fibroblast, hepatocyte, tumour cell. Mammalian cell lines for producing rAAV capsids in particular include a broad range of HEK293 cell lines. In a preferred embodiment, a mammalian cell as described herein is a mammalian cell for production of rAAV capsids. In one embodiment, there is provided the polynucleotide as described herein in a host cell, preferably in a mammalian cell. In a preferred embodiment, the host cell is a HEK293T cell.

In one embodiment, there is provided at least one expression vector comprising separate expression cassettes for each of the VP1, VP2 and VP3 capsid proteins, which capsid proteins, independently may be a variant, comprising a binding moiety, as described herein. In one embodiment, there is provided at least one expression vector comprising a separate expression cassette for a VP1 capsid protein and a separate expression cassette for the VP2 and VP3 proteins, wherein the VP1 capsid protein and/or both VP2 and VP3 capsid proteins, may be variants as described herein. The various expression cassettes for the different capsid protein variants as described herein can be present together on a single expression vector, or one or more of the expression cassettes for the different capsid proteins can be present on two or three separate expression vectors, each comprising one of the expression cassettes for the different capsid proteins. As will be understood, the use of separate expression cassettes for one or more of the different capsid proteins allows to produce rAAV capsids with an above-described modification/mutation in only some of capsid proteins, e.g. only in VP1 and not in VP2 and VP3 or vice versa, or it allows to produce rAAV capsids with different above-described modifications/mutations in the different capsid proteins. Expression vectors for separate expression of the various capsid proteins in mammalian cells are e.g. disclosed in Judd et al. (Mol Ther Nucleic Acids. 2012; 1: e54). Expression vectors for separate expression of the various capsid proteins in insect cells are disclosed in WO2022/253955.

The term “variant” as used herein refers to a nucleic acid sequence or amino acid sequence comprising one or more alterations as compared to a known or described reference sequence. In some embodiments, a variant sequence encodes a protein with altered characteristics or functionality as compared to the reference sequence. In other embodiments, a variant sequence encodes a protein with similar or the same functionality as compared to the reference sequence. In preferred embodiments, a variant has at least 70%, 75%, 80%, 85%, 90%, 95%, 96%, 97%, or 98%, preferably 99% sequence identity to a reference sequence.

In a preferred embodiment, there is provided an expression vector comprising a single expression cassette for expression of all three of the VP1, VP2 and VP3 capsid protein variants, comprising a binding moiety, as described herein, preferably from a single expression cassette. Expression vectors for expression of all three of the VP1, VP2 and VP3 capsid proteins from a single expression cassette in mammalian cells are e.g. disclosed in Clark et al. (Hum. Gene Ther. 1995, 6, 1329-134), Gao et al. (Hum. Gene Ther. 1998, 9, 2353-2362), Inoue and Russell (J. Virol. 1998, 72, 7024-7031), Grimm et al. (Hum. Gene Ther. 1998, 9, 2745-2760) and Xiao et al. (J. Virol. 1998, 72, 2224-2232). Expression vectors for expression of all three of the VP1, VP2 and VP3 capsid proteins from a single expression cassette in insect cells are e.g. disclosed in Urabe et al. (Hum. Gene Ther. 2002, 13:1935-1943), WO2007/046703, WO2015/137802 and WO2019/016349. As will be understood, expression of all three of the VP1, VP2 and VP3 capsid protein variants as described herein, from a single expression cassette allows to produce rAAV capsids comprising mutations/modifications as described herein in all three of its VP1, VP2 and VP3 capsid proteins.

In one embodiment, an expression vector for expression of an rAAV capsid as described herein comprises a nucleic acid comprising a suboptimal translation initiation codon for the VP1 capsid protein, wherein the suboptimal translation initiation codon is selected from CTG, ACG, TTG or GTG. In some embodiments, the suboptimal translation initiation codon is followed by an additional second codon encoding an amino acid selected from alanine, glycine, valine, aspartic acid and glutamic acid, preferably valine or alanine.

In one embodiment, the host cell further comprises an expression vector encoding an expression cassette flanked by at least one ITR, wherein the expression cassette comprises a nucleic acid molecule encoding at least one gene product.

### Composition

In a fourth aspect, there is provided a composition comprising an rAAV capsid as described herein, preferably wherein the composition is a pharmaceutical composition further comprising at least one pharmaceutically acceptable carrier.

In one embodiment, there is provided a composition comprising an rAAV capsid as described herein and a suitable excipient. Suitable excipients include, but are not limited to: a buffer, stabilizer, an antioxidant. In preferred embodiments the composition is used for treatment of (human) subjects. In some embodiments, the pharmaceutical composition comprises physiological buffers for buffering the composition to a pH of about 7.3-7.5. Exemplary physiological buffers include, but are not limited to: phosphate buffered saline (PBS), or tris (hydroxymethyl) aminomethane (Tris). Furthermore, a pharmaceutical composition may comprise further stabilizing agents including, but not limited to: sucrose, mannitol, trehalose, cyclodextrin, sodium chloride, potassium chloride, hydroxypropyl-beta-cyclodextrin (HP-BCD), or polysorbate. In a preferred embodiment, the pharmaceutical composition is suitable for systemic administration. More preferably the pharmaceutical composition is suitable for systemic administration using injection and/or infusion. In one embodiment, the pharmaceutical composition is suitable for intravenous and/or intraarterial injection and/or infusion.

rAAV Capsid or Composition for Use

In a fifth aspect there is provided a rAAV capsid as described herein, or a composition as described herein, for use as a medicament, preferably for use in the treatment of a condition of the central nervous system.

In one embodiment, there is provided a method of treating a condition of the CNS by administering an rAAV capsid as described herein, or composition as described herein.

In one embodiment, there is provided the use of an rAAV capsid as described herein in the manufacture of a medicament for the treatment of a condition of the CNS.

### Conditions of the Central Nervous System (CNS)

The terms “Central Nervous System” and “CNS”, as used herein refer to both the brain and the spinal cord.

The term a “condition of the central nervous system (CNS)” as used herein refers to a condition wherein the brain and/or spinal cord do not exert their function as compared to the brain and/or spinal cord of a healthy individual. The terms “a condition of the CNS”, “disease of the CNS” or “CNS disease” or “CNS disorder” may be used interchangeably. Diseases that also affect the brain and/or spinal cord next to having different symptoms can also be a “disease of the CNS”, or “CNS disease”. Such diseases include, for instance, neurological diseases which affect both the central nervous system and the peripheral nervous system.

In one embodiment, the brain and/or spinal cord function is impaired as compared to a healthy individual in a condition of the CNS as defined herein.

In a preferred embodiment, the condition of the CNS is caused by a genetic defect.

In a further embodiment, the genetic defect is a mutation in a gene and/or accumulation of dysfunctional or non-functional proteins when compared to a healthy individual.

Exemplary conditions of the CNS include, but are not limited to: Parkinson's Disease (PD); Parkinson disease 1, autosomal resistant (PARK1); Parkinson disease 4, autosomal dominant (PARK4); Multiple System Atrophy (MSA); Lewy Body Dementia (LBD); Huntington's Disease (HD); Spinocerebellar Ataxia 1 (SCA1); Spinocerebellar Ataxia 2 (SCA2); Spinocerebellar Ataxia 3 (SCA3); Primary Tauopathies; Pick's Disease (PiD); Progressive Supranuclear Palsy (PSP); Corticobasal Degeneration (CBD); Argyrophilic Grain Disease (AGD); Secondary Tauopathies; Chronic Traumatic Encephalopathy (CTE); Alzheimer's Disease (AD); Frontotemporal Dementia (FTD); Amyotrophic Lateral Sclerosis (ALS); Temporal Lobe Epilepsy (TLE); refractory Temporal Lobe Epilepsy (rTLE); Focal Cortical Dysplasia (FCD); Focal Cortical Dysplasia 2 (FCORD2); Smith-Kingsmore Syndrome (SKS); Spinal Muscular Atrophy 1 (SMA1; Werdnig-Hoffmann disease); Spinal Muscular Atrophy 2 (SMA2, Dubowitz disease); Spinal Muscular Atrophy 3 (SMA3, Kugelberg-Welander disease); Spinal Muscular Atrophy 4 (SMA4); Gaucher Disease (GD); Mucopolysaccharidosis 3A (MPS3A); Mucopolysaccharidosis 2 (MPS2); Mucopolysaccharidosis 1 (MPS1); Ceroid Lipofuscinosis, Neuronal, 1 (CLN1); Immunodeficiency 78 with autoimmunity and developmental delay (IMD78); Ceroid Lipofuscinosis, Neuronal, 3 (CLN3); Batten Disease; Ceroid Lipofuscinosis, Neuronal, 5 (CLN5); Ceroid Lipofuscinosis, Neuronal, 6 (CLN6); Ceroid Lipofuscinosis, Neuronal, 7 (CLN7); Ceroid Lipofuscinosis, Neuronal, 8 (CLN8); Canavan Disease (CAND); Ubiquitin-positive Frontotemporal Dementia (UP-FTD); Ceroid Lipofuscinosis, Neuronal, 11 (CLN11); Angelman Syndrome (AS); Intellectual Developmental Disorder, X-linked, Syndromic 13 (MRXS13); Rett Syndrome (RTT); Autism, X-linked 3 (AUTSX3); Encephalopathy, Neonatal Severe, due to MECP2 Mutations (ENS-MECP2); Intellectual Developmental Disorder, X-linked, Syndromic, Lubs type (MRXSL); GM1-Gangliosidosis 1 (GM1G1); GM1-Gangliosidosis 2 (GM1G2); GM1-Gangliosidosis 3 (GM1G3); Giant Axonal Neuropathy 1; Autosomal Recessive (GAN1); Citrullinemia (CTLN1); GM2-Gangliosidosis 1 (GM2G1); Tay-Sachs Disease; Fragile X Syndrome (FXS); Fragile X tremor/ataxia Syndrome (FXTAS); Cerebral Cavernous Malformations 4 (CCM4); Epilepsy, familial focal, with variable foci 1 (FFEVF1); Niemann-Pick Disease A (NPDA); Niemann-Pick Disease C (NPDC); Krabbe Disease (KRB); Mucopolysaccharidosis 1H (MPS1H); Epilepsy Metachromatic Leukodystrophy (MLD); Multiple Sulfatase Deficiency (MSD); Ceroid Lipofuscinosis, Neuronal, 2 (CLN2); Friedreich Ataxia (FRDA); Tuberous Sclerosis; Aromatic L-Amino Acid Decarboxylase Deficiency (AADCD); Pompe Disease; Mucopolysaccharidosis 3B (MPS3B); Alexander disease (ALXDRD); Dup15q syndrome; Developmental and epileptic encephalopathy 79 (DEE79); Epilepsy, childhood absence 5 (ECA5); Developmental and epileptic encephalopathy (DEE43); Prader-Willi syndrome (PWS); Phelan-McDermid Syndrome (PHMDS; PMS), Dravet Syndrome (DRVT); Lafora Disease (LD); Epilepsy, progressive myoclonic 2 (EPM2); Familial Hemiplegic Migraine 3 (FHM3); Generalized epilepsy with febrile seizures plus 2 (GEFSP2); Intractable childhood epilepsy with generalized tonic-clonic seizures (ICEGTC); Febrile seizures, familial, 3A (FEB3A); Developmental and epileptic encephalopathy 6B (DEE6B); Familial Hemiplegic Migraine 1 (FHM1); Episodic ataxia 2 (EA2); Developmental and epileptic encephalopathy 42 (DEE42); Familial Hemiplegic Migraine 2 (FHM2); Alternating hemiplegia of childhood 1 (AHC1); Fetal akinesia, respiratory insufficiency, microcephaly, polymicrogyria and dysmorphic facies (FARIMPD); Developmental and epileptic encephalopathy 98 (DEE98).

In a preferred embodiment, the condition of the CNS is selected from: Huntington's Disease (HD), Amyotrophic Lateral Sclerosis (ALS); Temporal Lobe Epilepsy (TLE); refractory Temporal Lobe Epilepsy (rTLE); Focal Cortical Dysplasia (FCD); Parkinson's Disease (PD); Parkinson disease 1, autosomal resistant (PARK1); Parkinson disease 4, autosomal dominant (PARK4); Multiple System Atrophy (MSA), Alzheimer's Disease (AD); Fragile X Syndrome (FXS); Fragile X tremor/ataxia Syndrome (FXTAS); and Dup15q syndrome.

In one embodiment, the rAAV capsid is administered systemically, preferably intravenously or intraarterially. In a preferred embodiment, the rAAV capsid is administered intravenously or intraarterially at a site close to the BBB.

### Gene Therapy

In a sixth aspect, there is provided a rAAV capsid as described herein, or a composition as described herein, for use in gene therapy.

The term “gene therapy” as used herein refers to a therapy that uses one or more gene products as described herein to treat, prevent or cure a disease or medical disorder. In one embodiment, the treatment, prevention or cure is long-lasting and sustained and follows upon a single administration of the gene therapy.

In one embodiment, there is provided a method of gene therapy comprising the step of administering an effective amount of an rAAV capsid as described herein, or a composition comprising the rAAV capsid as described herein, to a subject in need of gene therapy.

In one embodiment, the gene therapy is for the treatment of a condition of the CNS as defined herein above.

### Method of Manufacturing

In a seventh aspect, there is provided a method of manufacturing an rAAV capsid as described herein, the method comprising the steps of: I) culturing a host cell comprising: i) an expression vector for expression of a rAAV capsid as described herein, and; ii) an expression vector encoding an expression cassette flanked by at least one AAV inverted terminal repeat (ITR), comprising a nucleic acid molecule encoding at least one gene product; said host cell preferably further comprising a nucleotide sequence encoding a parvoviral replication (Rep) protein under conditions such that the rAAV capsid is produced; and, II) recovery of the rAAV capsid, preferably wherein recovery of the rAAV capsid comprises at least one of: affinity-purification of the rAAV capsid using an immobilized anti-AAV antibody, preferably a single chain camelid antibody or a fragment thereof, or filtration using a filter having a nominal pore size of 30-70 nm.

In general, suitable methods for producing an rAAV capsid as described herein in mammalian or insect host cells, and means therefore (such as expression constructs for expression of AAV rep proteins), are described, for mammalian cells in: Clark et al. (Hum. Gene Ther. 1995, 6, 1329-134), Gao et al. (Hum. Gene Ther. 1998, 9, 2353-2362), Inoue and Russell (J. Virol. 1998, 72, 7024-7031), Grimm et al. (Hum. Gene Ther. 1998, 9, 2745-2760), Xiao et al. (J. Virol. 1998, 72, 2224-2232) and Judd et al. (Mol Ther Nucleic Acids. 2012; 1: e54), and for insect cells in: Urabe et al. (Hum. Gene Ther. 2002, 13:1935-1943), WO2007/046703, WO2007/148971, WO2009/014445, WO2009/104964, WO2011/122950, WO2013/036118, WO2015/137802, WO2019/016349, WO2021/198508, WO2021/198510 and WO2022/253955, all of which are incorporated herein in their entirety.

AAV sequences that may be used as described herein for the production of an rAAV capsid as described herein in insect cells can be derived from the genome of any AAV serotype. Generally, the AAV serotypes have genomic sequences of significant homology at the amino acid- and nucleic acid levels, provide an identical set of genetic functions, and produce capsids which are essentially physically and functionally equivalent, and replicate and assemble by practically identical mechanisms. For the genomic sequence of the various AAV serotypes and an overview of the genomic similarities see e.g. GenBank Accession number U89790; GenBank Accession number J01901; GenBank Accession number AF043303; GenBank Accession number AF085716; Chlorini et al. (J. Vir. 1997, 71:6823-33); Srivastava et al. (J. Vir. 1983, 45:555-64); Chlorini et al. (J. Vir. 1999, 73:1309-1319); Rutledge et al. (J. Vir. 1998, 72:309-319); and Wu et al. (J. Vir. 2000, 74:8635-47). Preferably the AAV ITR sequences for use in the context of the present invention are derived from AAV1, AAV2, AAV4 and/or AAV7, more preferably AAV2. Likewise, the Rep (Rep78/68 and Rep52/40) coding sequences are preferably derived from AAV1, AAV2, AAV4 and/or AAV7.

AAV Rep and ITR sequences are particularly conserved among most serotypes. The Rep78 proteins of various AAV serotypes are e.g. more than 89% identical and the total nucleotide sequence identity at the genome level between AAV2, AAV3A, AAV3B, and AAV6 is around 82% (Bantel-Schaal et al., J. Virol., 1999, 73(2):939-947). Moreover, the Rep sequences and ITRs of many AAV serotypes are known to efficiently cross-complement (i.e., functionally substitute) corresponding sequences from other serotypes in production of AAV particles in mammalian cells. US2003148506 reports that AAV Rep and ITR sequences also efficiently cross-complement other AAV Rep and ITR sequences in insect cells. Modified “AAV” sequences also can be used in this context, e.g. for the production of rAAV vectors in insect cells. Such modified sequences e.g. include sequences having at least about 70%, at least about 75%, at least about 80%, at least about 85%, at least about 90%, at least about 95%, or more nucleotide and/or amino acid sequence identity to an AAV1, AAV2, AAV3, AAV4, AAV5, AAV6, AAV7, AAV8, AAV9, AAV10, AAV11, AAV12 or AAV13 ITR or Rep can be instead of place of wild-type AAV ITR or Rep sequences

In one embodiment, a mammalian cell for producing an rAAV capsid as described herein is selected from cell lines among any mammalian species, including, but not limited to: A549, WEHI, 3T3, 10T1/2, BHK, MDCK, COS 1, COS 7, BSC 1, BSC 40, BMT 10, VERO, WI38, HeLa, a HEK 293 cell, Saos, C2C12, L cells, HT1080, HepG2 and primary fibroblast, hepatocyte and myoblast cells derived from mammals including human, monkey, mouse, rat, rabbit, and hamster. The selection of the mammalian species providing the cells is not a limitation of this disclosure; nor is the type of mammalian cell, i.e., fibroblast, hepatocyte, tumour cell. Mammalian cell lines for producing rAAV capsids in particular include a broad range of HEK293 cell lines, of which the HEK293T cell line is preferred.

Insect cell lines for producing an rAAV capsid as described herein can be any cell line that is suitable for the production of heterologous proteins. Preferably the insect cell allows for replication of baculoviral vectors and can be maintained in culture, more preferably in suspended culture. In a preferred embodiment, the insect cell allows for replication of recombinant parvoviral vectors, including rAAV vectors. For example, the cell line used can be from Spodoptera frugiperda, Drosophila, or mosquito, including Aedes albopictus-derived cell lines. In a preferred embodiment, the insect cell or cell line is derived from insect species which are susceptible to baculovirus infection, including but not limited to: S2 (CRL-1963, ATCC), Se301, SelZD2109, SeUCR1, Sf9, Sf900+, Sf21, BTI-TN-5B1-4, MG-1, Tn368, HzAm1, Ha2302, Hz2E5, High Five (Invitrogen, CA, USA) and expresSF+® (U.S. Pat. No. 6,103,526; Protein Sciences Corp., CT, USA).

The rAAV capsids that are released into the supernatant of the mammalian or insect cell culture can be recovered and/or purified using suitable techniques which are known to those of skill in the art. In one embodiment, monolith columns (e.g., in ion exchange, affinity or IMAC mode), chromatography (e.g., capture chromatography, fixed method chromatography, and expanded bed chromatography), centrifugation, filtration and/or precipitation, are used for purification and concentration. These methods may be used alone or in combination. In one embodiment, capture chromatography methods, including column-based or membrane-based systems, are utilized in combination with filtration and precipitation. Suitable precipitation methods, including, but not limited to, chromatography methods utilizing polyethylene glycol (PEG) 8000 and NH3SO4, can be readily selected by one of skill in the art. Thereafter, the precipitate can be treated with enzymes, including but not limited to: benzonase, and purified using suitable techniques. In addition, recovery preferably comprises the step of affinity-purification of the rAAV capsid using an anti-AAV antibody, preferably an immobilised antibody. The anti-AAV antibody preferably is a monoclonal antibody. A particularly suitable antibody is a single chain camelid antibody or a fragment thereof as e.g. obtainable from camels or llamas (see e.g. Muyldermans, Biotechnol. 2001, 74:277-302). The antibody for affinity-purification of rAAV capsids preferably is an antibody that specifically binds an epitope on an rAAV capsid protein, whereby preferably the epitope is an epitope that is present on capsid protein of more than one AAV serotype. E.g. the antibody may be raised or selected on the basis of specific binding to AAV6 capsid but at the same time also it may also specifically bind to rAAV9 capsids.

### DESCRIPTION OF THE FIGURES

FIG. 1. Schematic diagram depicting generation of rAAV capsids with different binding moieties.

A) plMRAC (Insert-cell & MRna-based Aav Capsid library) library backbone comprising a randomly degenerated sequence (rainbow box). B) Rolling Circle amplification and TelN digestion: amplification of the library and generation of suitable templates for rAAV library production. C) Transfection and baculovirus infection of cells, generating rAAV capsids.

FIG. 2. Screening for hTfR1-interacting capsid variants.

A) In vitro screening of capsid library. B) Analysis of the capsid cDNA output by next-generation sequencing (NGS) for random codon/peptide enrichment within the VR-VIII loop. Distribution of the random peptide library is mapped in comparison to the β capsid library input and the capsid library output from in vitro evolution in naïve HEK293T cells. C) Capsid variants found to be enriched in hTfR1-overexpressing cells only.

FIG. 3. Schematic Diagram Illustrating the Single-Plex Method for Hit Discovery and Identification of AAVCapX Hits Interacting with TfR1.

A) Capsid variant expression in insect cells. B) Assessment of Nluc expression in cells harbouring TfR1 or a mock receptor (human Carbonic Anhydrase 4 (hCA4)). C) Fold enrichment of the different capsid variants over mock receptor (hCA4). D) Capsid variants with highest enrichment scores for TfR1. E) Phylogenetic analysis of hTfR1-engaging capsid variants.

FIG. 4. Dose-Range and Tropism Confirmation of TfR1-Interacting AAVCapX Variants.

A) Ratio of human TfR1 (Nluc) over mock receptor reporter (Fluc) for four CapX variants, compared to two control capsids (Capctrl and Cap BI hTfR1), at increasing MOI (5E+2, 5E+3, or 5E+4 GC/cell). B) Ratio of macaque (macaca fascicularis) TfR1 reporter (Nluc) over mock receptor reporter (Fluc) for four CapX variants, compared to two control capsids, at increasing MOI (5E+2, 5E+3, or 5E+4 GC/cell). B) Ratio of murine (mus musculus) TfR1 reporter (Nluc) over mock receptor reporter (Fluc) for four CapX variants, compared to two control capsids, at increasing MOI (5E+2, 5E+3, or 5E+4 GC/cell).

FIG. 5. Histology readout in murine brain tissue.

The top two brain sections represent eGFP Immunohistochemistry in a sagittal section from one representative humanized mouse (out of four) injected IV with either AAV-X230 or AAV9 capsid. The below four sections represent the eGFP IHC of mice injected with AAV-X230, highlighting different relevant brain regions such as isocortex, striatum, hippocampus and cerebellum.

FIG. 6. Transgene expression in different brain regions.
GFP specific primers and probes were used to measure transgene expression in different brain regions (rostral, caudal, and cerebellum with brainstem). Transgene levels in mice injected with AAV-X230 are between 11 and 14-fold higher compared to AAV9.

FIG. 7. BBB Transwell model.

Either AAV9 or AAV-X230 was used to transduce an apically positioned brain microvascular endothelial cell layer (BMEC) of the transwell model. The basolateral layer of the transwell model contained primary human astrocytes. 24 Hours after transduction, vDNA was measured in the basolateral media. Subsequently, the basolateral media was used to transduce HEK293T recipient cells to assess the retained potency of the BBB-crossing capsids.

### EXAMPLES

### Introduction

In this study, a library and screening platform was used to generate and screen for rAAV capsids capable of utilizing human Transferrin receptor protein 1 (hTfR1) to cross the BBB and achieve widespread brain transduction via systemic administration. Conventional capsid library evolution strategies are less suited to this end as they would yield capsid candidates with random properties. Therefore and in vitro screening strategy was used to simplify and rationally bias the capsid library evolution towards hTfR1 usage.

### 1. Example 1: Generation of the Capsid Library and Its Application in In Vitro Screening for hTfR1-Dependent BBB-Penetrating Capsids

### 1.1 Methods

### 1.1.1 BRAIN-X AAV Capsid Library Production

DNA libraries were constructed by cloning double-stranded DNA fragments into a digested recipient pIMRAC plasmid using the Gibson assembly method (NEBuilder HiFi DNA Assembly Master Mix). Plasmid propagation was performed using rolling circle amplification (RCA) with EquiPhi29™ DNA Polymerase (ThermoFisher). The resulting DNA product was digested with TelN protelomerase, which targets TelN recognition sites in the plasmid backbone.

The resulting DNA was transfected into Sf+ cells at a ratio of 0.07 pg per cell. Three days post-transfection, the cells were infected with a baculovirus encoding AAV replicase. Seventy-two hours post-infection, the cells were lysed, and the rAAV capsid library was harvested and purified using POROS CaptureSelect AAVX Affinity Resin.

The rAAV capsid library was then titrated using qPCR with primers/probes binding to the hr4b enhancer. The presence and relative abundance of viral proteins were analyzed by SDS-PAGE. Additionally, viral DNA was isolated from the purified product using the PureLink Viral RNA/DNA Mini Kit and used as a template to amplify the library insertion site for Sanger sequencing, confirming the transfer of the library from the cloned plasmid to the final capsid library.

### 1.2 Results

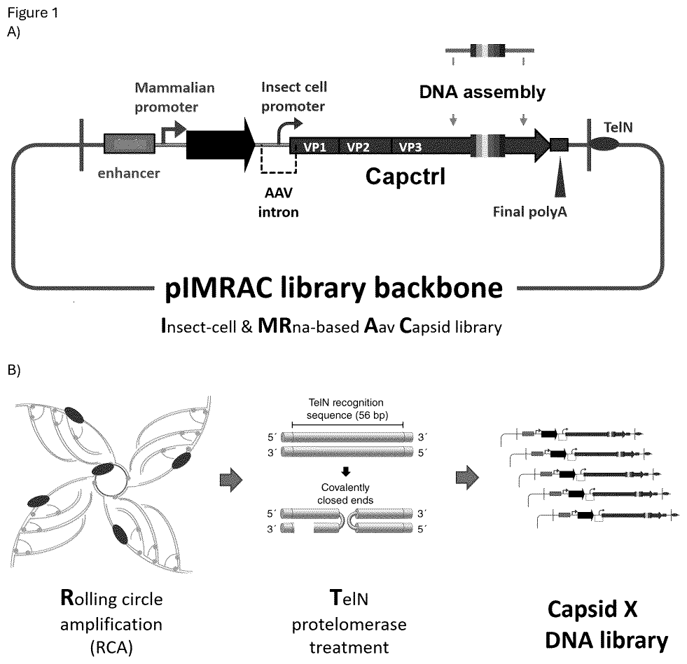

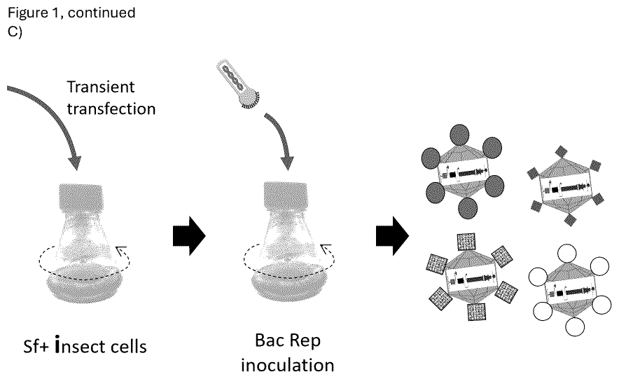

A DNA fragment with a randomly degenerated sequence (indicated as a rainbow box in FIG. 1A) was synthesized and cloned into the VR-VIII loop of a rAAV9 capsid sequence [(capctrl)—SEQ ID NO: 298] in pIMRAC (Insect-cell & mRNA-based AAV Capsid library) (FIG. 1A). Two versions of the library were created: Library #1 contains a 7× degenerated NNK codon sequence between Q588 and A596, while Library #2 includes codons encoding D587, G588, and P589, followed by 5× degenerated NNK codons and R595. Immediately after cloning, the DNA mixture was subjected to rolling circle DNA amplification (RCA) and TelN digestion to generate the capsid-X DNA genome library (FIG. 1B). This library was then used in transfection and infection experiments (FIG. 1C). Following baculovirus Rep (Bac Rep) transactivation, the transfected insect cells produced rAAV capsids. These capsids have random peptide insertions in the VR-VIII loop derived from the DNA genome previously transfected into the cells, and the same DNA genome is also encapsidated by the newly produced capsids.

### 2. Example 2: Confirmation of Library Hit Interactions With hTfR1 via a Single-Plex Discovery Assay

### 2.1 Methods

2.1.1 rAAV Capsid Library In Vitro Screening

The rAAV capsid libraries were screened in vitro using a transient expression assay in HEK293T cells, overexpressing human Transferrin receptor protein 1 (hTfR1) along with firefly Luciferase and mScarlet reporters. Cells were transduced with the rAAV capsid library at an MOI of 1e5 and co-infected with wild-type adenovirus 5 (Ad5). After 48 hours, the transfected and transduced cell subpopulation was sorted using FACS, lysed, and total RNA was isolated using the AllPrep DNA/RNA Micro Kit, followed by cDNA synthesis and PCR amplification of the library insertion site. Sanger sequencing was employed to evaluate the presence of the library in the resulting PCR product, which was subsequently re-cloned into the vector plasmid to produce the β capsid library for a second in vitro iteration.

### 2.1.2 NGS Sample Preparation, Sequencing, and Analysis

After in vitro screening, the fragment containing the library was PCR amplified from: 1) the vDNA from the β capsid library used for transduction in the second iteration (input), 2) the cDNA from hTfR1-overexpressing cells transduced with the β capsid library, and 3) the cDNA from HEK293T cells, with baseline hTFR1 expression, transduced with the β capsid library. The PCR amplicons were used for Illumina library preparation (TruSeq DNA Nano Kit), and the resulting libraries were sequenced on the NovaSeq6000 sequencer with paired-end reads of 151 nt, aiming for at least 5 million paired-end reads per sample.

Paired reads were merged with Pear (v0.9.6) and translated into all possible reading frames using seqtk. An in-house developed Python script was used to identify the amino acid and nucleotide sequences of the capsid variants and their abundance in each sample. Raw counts of each variant were normalized to counts per million (CPM) to account for differences in sequencing depth across samples. The enrichment score of each capsid variant in the hTfR1-expressing cells was calculated by dividing the CPM in hTfR1-expressing cells by the CPM in the input β capsid library. Similarly, to account for hTfR1-binding specificity, the CPM of each capsid variant in the hTfR1-expressing cells was divided by the CPM in HEK293T cells (naïve cells). For hit selection, capsid variants that were enriched ≥10-fold in hTfR1 relative to the β capsid library input and ≥5-fold relative to HEK293T naïve cells were cloned into a plasmid backbone suitable for small-scale production in Sf+ cells and single-plex validation.

### 2.1.4 Single-Plex Hit Discovery Assay

HEK293T cells were seeded and reverse transfected with either a plasmid (pOX) encoding hTfR1, mScarlet, and FLuc reporters, or with a plasmid expressing both mScarlet and firefly luciferase (FLuc) reporters but with a mock receptor. Two days after transfection, cells were transduced with each capsid variant. Forty-eight hours post-transduction, cells were lysed, and FLuc and NLuc were measured using the Nano-Glo Dual-Luciferase Reporter Assay System. The enrichment score of each capsid variant in the hTfR1-transfected cells was calculated by dividing the NLuc readout in the hTfR1-transfected cells relative to the NLuc readout in cells transfected with an unrelated receptor (hCA4). Initially, cells were transduced with a single (volumetric) MOI; subsequently, cells were transduced with three increasing MOIs (5E+2, 5E+3, or 5E+4 GC/cell). All experiments were performed in triplicate with n=2.

### 2.2 Results

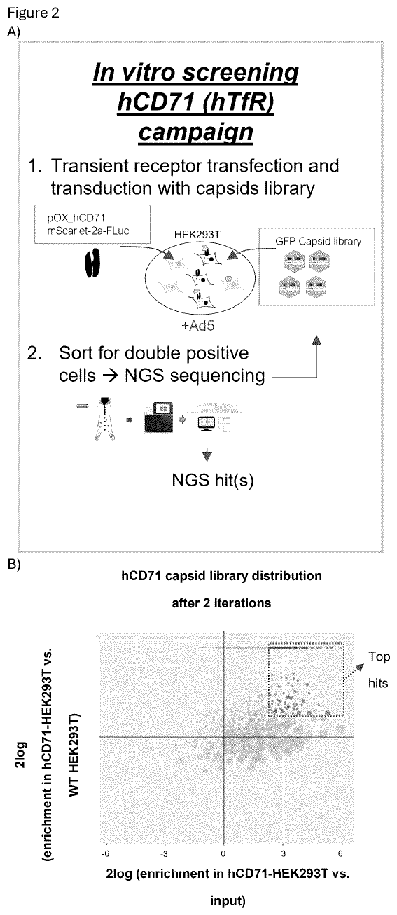

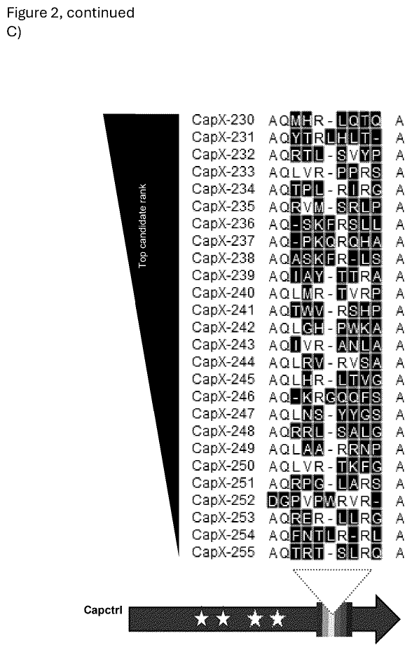

HEK293T cells overexpressing hTfR1 were transduced with the 1st generation (a) capsid library and wild-type adenovirus (Ad5) (FIG. 2A). The use of Ad5 amplified the cDNA expression from the capsid genome library in the target cells, which was then used to generate the 2nd generation (B) capsid library for the next iteration. After 2 iterations, the capsid cDNA output was analyzed by next-generation sequencing (NGS) for random codon/peptide enrichment within the VR-VIII loop. During enrichment analysis, the distribution of the random peptide library was mapped in comparison to the β capsid library input and the capsid library output from in vitro evolution in naïve HEK293T cells only (FIG. 2B). As a result, several variants of AAV9 harboring random peptide insertions were observed to be enriched in hTfR1 overexpressing cells only (FIG. 2C).

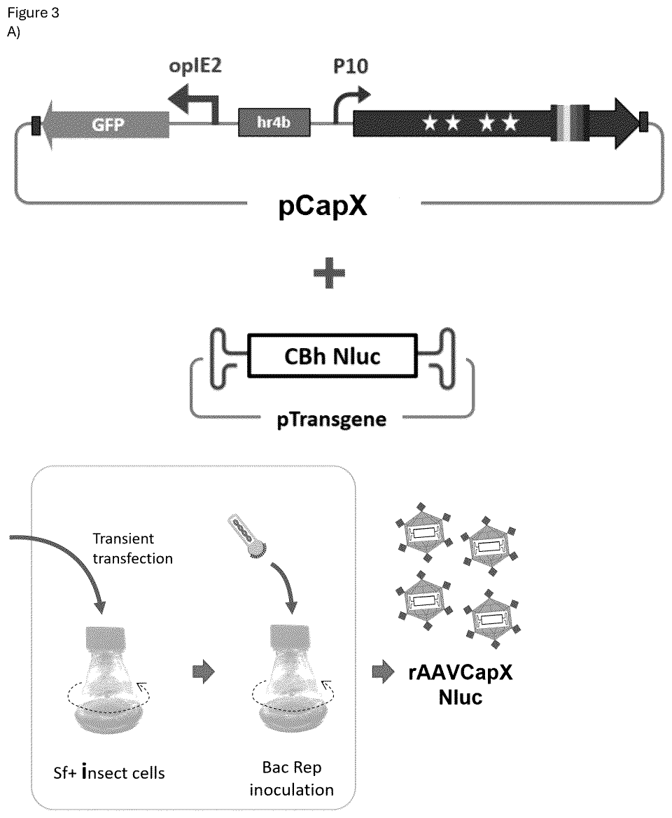

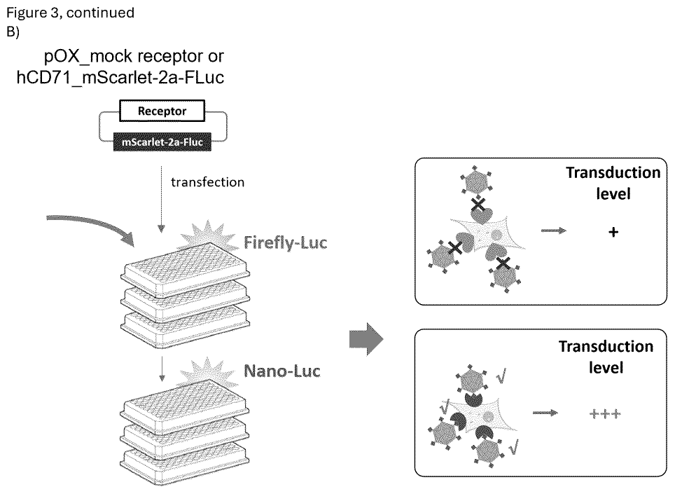

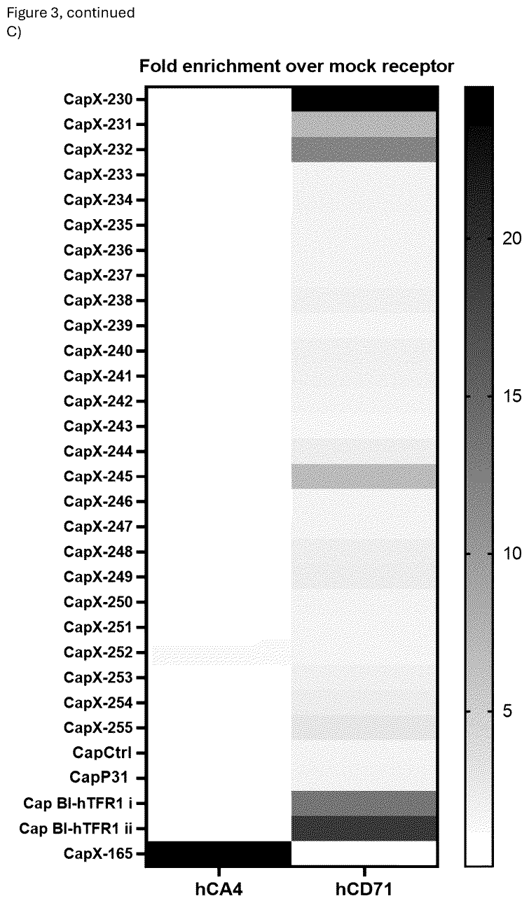

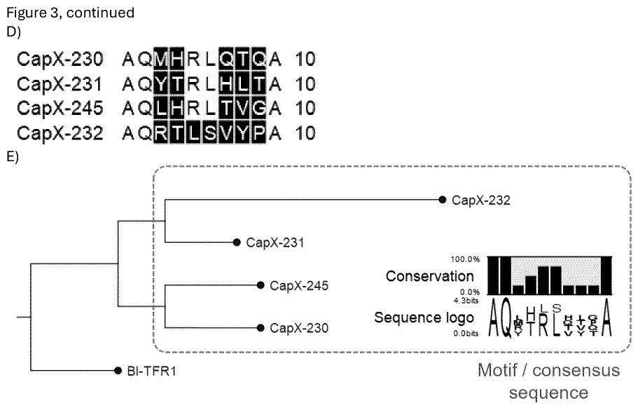

Subsequently, the top 26 hTfR1 engager peptide variants were individually cloned into an rAAV9 variant sequence within the pCapX backbone (FIG. 2C). Each pCapX hit was then co-transfected with the nano-luciferase (Nluc) reporter into insect cells. Following Bac Rep transactivation, viable rAAV particles were produced, each displaying a novel capsid variant (rAAVCapX) (FIG. 3A).

To confirm and score the engagement of these novel capsid variants with the hTfR1 transmembrane receptor, a single-plex hit discovery assay was developed. This assay is based on the principle that exogenous expression of the co-opted transmembrane receptor in mammalian cells selectively improves the potency of rAAV with an engineered capsid that engages with the receptor, compared to naïve AAV9, at a low multiplicity of infection (MOI) (Shay et al., 2024, supra). In this assay, hTfR1 or a mock transmembrane receptor (hCA4) was transiently overexpressed in HEK293T cells via pOX transfection. The pOX plasmid also expressed equimolar mScarlet and Firefly luciferase to represent transfection efficiency. After transfection, each AAVCapX was added, and the cells were assessed for Nluc expression. AAVCapX variants that can co-opt hTfR1 as an entry receptor are expected to show increased transduction potency compared to those that do not engage with the receptor (FIG. 3B).

The transient overexpression of hTfR1 in target cells improved the potency of an rAAV9 variant displaying the BI-TFR1 peptide (Cap BI-TFR1), a known hTfR1 engager (Huang et al, supra), by approximately ˜10-fold, thereby validating the assay (FIG. 3C). Several rAAVCapX variants displaying the hTfR1 peptide hits were confirmed (FIG. 3C) to interact with hTfR1 overexpression, showing a ˜10-20-fold increase in potency. Analyzing the top 4 of hCD71/hTfR-interacting peptide hits, a conserved 7-mer peptide motif (XXRLXXX (SEQ ID NO: 297)) was identified (FIG. 3D). Phylogenetic analysis of the total hCD71/hTfR-engaging peptide hits demonstrated distinct sequence to the BI-TFR1, clustering around the XXRLXXX motif (SEQ ID NO: 297) (FIG. 3E).

### 3. Example 3: Dose-Dependent and Docking-Site Validation of AAVCapX Interaction With the hTfR1 Receptor

### 3.1 Methods

3.1.1. Production of rAAVCapX variants

Top-selected rAAVCapX variants were cloned into a pCapX plasmid suitable for AAV production in Sf+ cells when co-transfected with a pTransgene plasmid containing nano-luciferase (NLuc) under a ubiquitous promoter (CBh). Using this method, each capsid variant was produced at a small scale. Specifically, pCapX and pTransgene DNA was transfected into 1.5×10\hat{ }7 ExpresSF+ cells using Cellfectin II transfection reagent (ThermoFisher). Seventy-two hours post-transfection, fresh baculovirus expressing AAV2 Replicase was inoculated into the cells at 1% of the final volume (40 ml). Seventy-two hours post-baculovirus infection, the rAAV batches were harvested by adding 10× lysis buffer (1.5 M NaCl, 0.5 M Tris-HCl, 1 mM MgCl2, 10% Triton X-100, pH 8.5) and lysed for 1 hour at 28° C. Genomic DNA was digested by benzonase treatment for 1 hour at 37° C. Cell debris was removed by centrifugation for 15 minutes at 1900×g, after which the supernatant containing the rAAVCapX capsid variants was stored at 4° C.

rAAVCapX capsids were purified from crude lysates using a batch binding protocol involving AAVx affinity resin (ThermoFisher). Briefly, crude cell lysates were added to resin washed with 0.2 M HPO4 buffer (pH 7.5). The samples were incubated for 2 hours at room temperature with gentle mixing. After binding, the resin was washed with 0.2 M HPO4 buffer (pH 7.5), and the bound vectors were eluted by adding 0.2 M Glycine (pH 2.5). The pH of the eluate was immediately neutralized by adding 0.5 M Tris-HCl (pH 8.5). The purified rAAVCapX variant capsids were stored at −20° C.

### 3.2 Results

To validate the interaction between the co-opted transmembrane receptor and the engineered rAAV capsid, a dose-dependent, multiplicity of infection (MOI)-based rAAV transduction study was conducted. In this study, rAAVCapX capsid variants with the highest enrichment scores were selected for individual production on a larger scale, with the goal of packaging the Nluc reporter. Each rAAVCapX capsid variant was applied in a dose-dependent manner to HEK293T cells that were transfected with equimolar amounts of either the pOX mock receptor, or hTfR1, macaque TfR1 (mfTfR1), or murine TfR1 (mmTfR1). The expression of the Nluc reporter, normalized to the Fluc expression from the transfected pox, was measured to assess the transduction potency of the respective rAAV capsids.

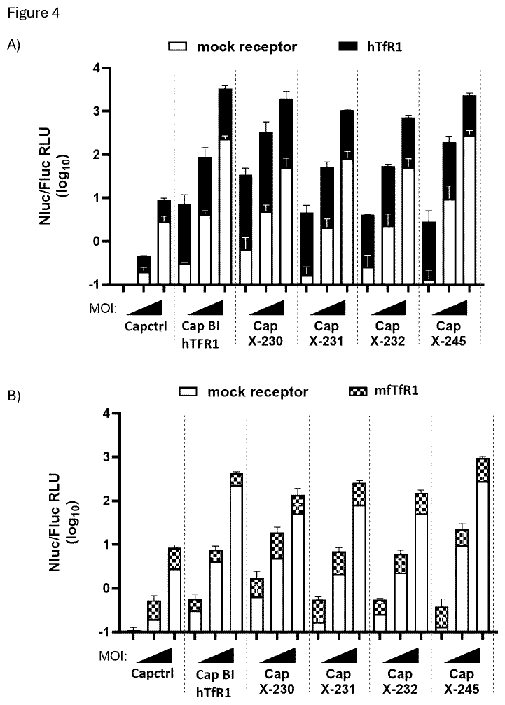

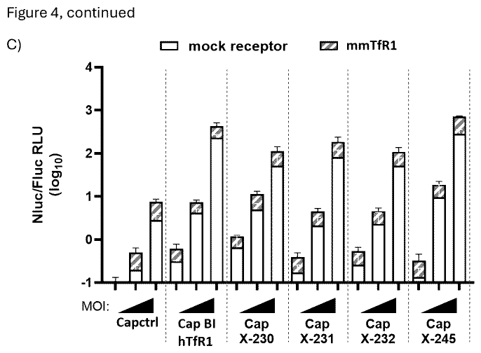

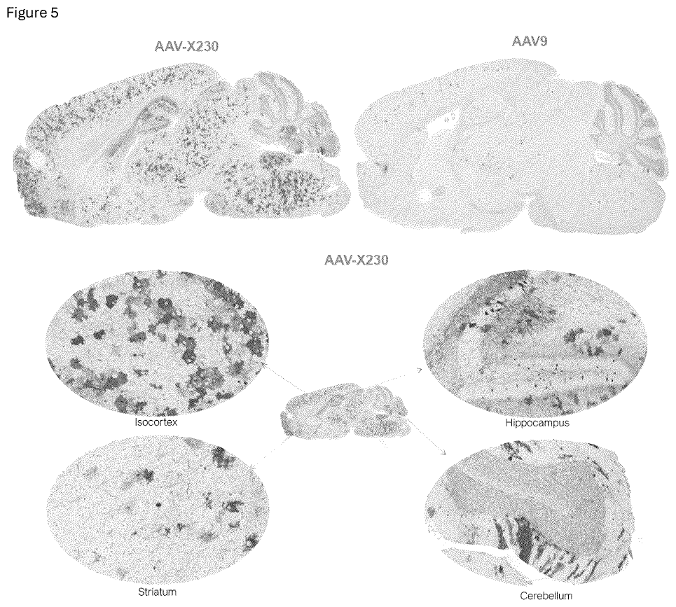

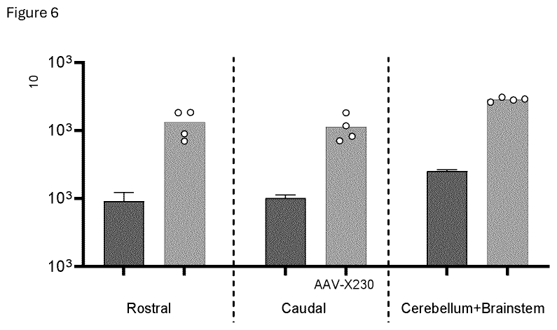

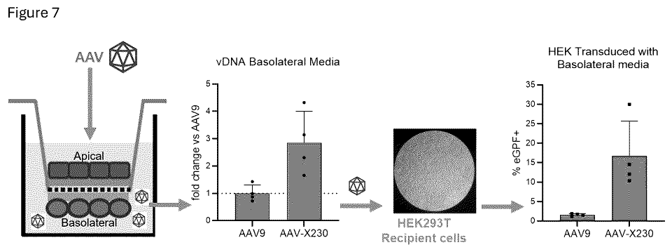

As expected, the positive control (Cap BI hTFR1) and the rAAVCapX variants shows a dose-dependent enhancement in transduction potency in cells overexpressing hTfR1 (FIG. 4A). Intriguingly, the TfR1 interaction of rAAVCapX variants was found to be species-restricted (FIG. 4B and C), with much higher interaction rates in hTfR1-expressing cells compared to mfTfR1 and mmTfR1. Interestingly, the rAAVCapX variants share this feature with Cap BI hTFR1, a capsid that is known to cross the BBB. This suggests that the rAAVCapX variants may interact with a similar binding domain.

### 4. Example 4: Systemic Intravenous Delivery of hTfR1-Interacting rAAVCapX Variants Crosses the BBB and Transduces the Brain In Vivo

### 4.1 Methods

hTfR1-interacting rAAVCapX capsid variants were identified and produced at a larger scale, carrying an eGFP transgene under regulation of a ubiquitous promoter (Alt promoter). A transgenic mouse model expressing the hTfR1 receptor was used to validate our capsid in vivo. Either AAV9 wild-type (SEQ ID NO: 302) or our AAV capsid QX-230 carrying the CapX-230 peptide engrafted on the AAV9-like backbone capctrl (SEQ ID NO: 298) were delivered IV at a dose of 1.5e13 vg/kg. Each group consisted of 4 mice which were monitored for four weeks for any adverse effects until terminal anesthesia and cardiac perfusion with ice-cold heparinized 0.9% saline. This was followed by brain harvesting for immunohistochemistry (IHC) analysis and molecular readouts such as vector DNA and vector mRNA, measured by qPCR. For molecular analysis, tissue samples were pulverized and DNA and RNA was isolated using AllPrep DNA/RNA Mini (Qiagen). Total RNA was reverse transcribed using Maxima First Strand cDNA synthesis Kit for RT-PCR (Thermo Scientific).

### 4.2 Results

The hTfR1-interacting rAAVCapX capsid AAV QX-230 was shown to successfully transduce brain cells, resulting in significant transduction in the brain tissue of treated animals compared to the control group treated with AAV9, according to immunohistochemistry against eGFP (FIG. 5). AAV QX-230 transduction levels, measured by means of eGFP mRNA levels by qPCR, are between 11- and 14-fold higher compared to AAV9 wild type depending on the brain region, being slightly more enriched in the rostral section (FIG. 6). This experiment demonstrates the potential of using hTfR1-interacting rAAV capsid variants for targeted gene delivery across the BBB, paving the way for potential therapeutic applications in treating neurological diseases.

### 5. Example 5: hTfR1-Interacting rAAVCapX Capsid Variants Can Cross the Human BBB as Assessed Using a Human BBB In Vitro Model

### 5.1 Methods

hTfR1-interacting rAAVCapX capsid variants are identified and produced on a larger scale. A human BBB transwell model was constructed by seeding hCMEC/D3 cells at 2×10\hat{ }5 cells/cm² on the top side of a 0.4-μM PET membrane pre-coated with collagen, and human primary astrocytes at 6×10\hat{ }4 cells/cm² on the bottom side of the membrane pre-coated with poly-l-lysine. This model was incubated for 24 hours, after which the hCMEC/D3 cells were seeded on the top side and the culture was maintained with media changes every 2-3 days for total of 11 days. Heparin sulfate was omitted from the media to avoid interference with rAAV association. Either AAV9 wild-type (SEQ ID NO: 302) or an AAV9 wild-type capsid sequence wherein the CapX-230 peptide (SEQ ID NO: 1) was inserted (capsid QX-270 (SEQ ID: 306) were introduced on the apical side of the transwell (top chamber).

Twentyfour hours after transduction, vDNA was measured in basolateral media. Next, the basolateral media used to transduce HEK293T recipient cells in order to assess the retained potency of the BBB-crossing capsids.

### 5.2 Results

The hTfR1-interacting rAAVCapX capsid AAV QX-270 comprising the X230 peptide insert was shown to cross the transwell BBB model 3-fold better as compared to an AAV capsid formed by an AAV9 wild type capsid protein. This was quantified using the vDNA levels measured in the media of the basolateral layer (FIG. 7). Additionally, the viral particles present in the basolateral layer retain their potency, as was evidenced by the transduction levels in HEK293T recipient cells upon incubation of the media that was in contact with the basolateral layer (FIG. 7). These results support the potential use of hTfR1-interacting rAAV capsid variants for targeted gene delivery across the human BBB, providing insights into developing treatments for neurological conditions.

1. A recombinant adeno-associated virus (rAAV) capsid comprising a binding moiety, wherein the binding moiety confers to the rAAV capsid a phenotype of increased interaction with Transferrin receptor protein 1 (TfR1) as compared to a control rAAV capsid not comprising the binding moiety.

2. The rAAV capsid according to claim 1, wherein the rAAV capsid presents at least one of the following phenotypes upon systemic administration:
i) a phenotype of increased blood brain barrier (BBB) crossing as compared to a control rAAV capsid not comprising the binding moiety; and/or
ii) a phenotype of increased transduction of cells in the central nervous system (CNS) as compared to a control rAAV capsid not comprising the binding moiety.

3. The rAAV capsid according to claim 1, wherein at least part of the binding moiety is inserted between two consecutive amino acids of a capsid protein.

4. The rAAV capsid according to claim 1, wherein the binding moiety is comprised in a variable region (VR) of a surface loop of the rAAV capsid, preferably in at least one of the VR-I, VR-II, VR-III, VR-IV, VR-V, VR-VI, VR-VII, VR-VIII and/or VR-IX, preferably in the VR-IV and/or VR-VIII, more preferably in the VR-VIII.

5. The rAAV capsid according to claim 1, wherein the binding moiety comprises an amino acid insert between two consecutive amino acids present within amino acids 580-595, preferably amino acids 585-590, more preferably amino acids 588 and 589 of an AAV9 capsid protein, or in an analogous position of a capsid protein of an AAV capsid serotype selected from the group comprising: AAV1, AAV2, AAV3A, AAV3B, AAV4, AAV5, AAV6, AAV7, AAV8, AAV10, AAV11, AAV12, AAV13 and AAVrh10, or modified versions thereof, preferably rAAV6, or modified versions thereof.

6. The rAAV capsid according to claim 1, wherein the amino acid insert consists of 2, 3, 4, 5, 6, 7, 8, 9, 10, 11, 12, 13, 14, 15 or more amino acids, preferably 5-9 amino acids, more preferably 7 amino acids.

7. The rAAV capsid according to claim 1, wherein the binding moiety comprises at least 4, 5, 6, or 7 contiguous amino acids of an amino acid sequence X₁X₂X₃X₄X₅X₆X₇X₈ (SEQ ID NO: 295), wherein:
X₁ is absent, or selected from: M, Y, R, L, T, A, I, P and F;
X₂ is selected from: H, T, V, P, S, A, M, W, G, R, K, N and F;
X₃ is selected from: R, L, M, K, Y, V, H, S, A, G, P and T;
X₄ is absent, or selected from: L, F, Q, G and W;
X₅ is selected from: L, H, S, P, R, T, A, Q and Y;
X₆ is absent, or selected from: Q, L, V, P, I, R, S, T, W, N, Y, A and K;
X₇ is selected from: T, Y, R, L, H, K, S, V, F, G and N, and;
X₈ is absent, or selected from: Q, P, S, G, L and A;
optionally wherein the amino acid at position 587 is mutated to D, and the amino acid at position 588 is mutated to G.

8. The rAAV capsid according to claim 1, wherein:
the binding moiety comprises a consensus sequence AQX₁X₂X₃X₄X₅X₆X₇X₈ (SEQ ID NO: 296, wherein
X₁ is absent, or selected from: M, Y, R, L, T, A, I and F;
X₂ is selected from: H, T, V, P, S, A, M, W, G, R, K, N and F;
X₃ is selected from: R, L, M, K, Y, V, H, S, A, G and T;
X₄ is absent, or selected from: L, F, Q and G;
X₅ is selected from: L, H, S, P, R, T, A, Q and Y;
X₆ is absent, or selected from: Q, L, V, P, I, R, S, T, W, N, Y, A and K;
X₇ is selected from: T, Y, R, L, H, K, S, V, F, G and N, and;
X₈ is absent, or selected from: Q, P, S, G, L and A;
or, wherein the binding moiety comprises the amino acid sequence DGPVPWRVR (SEQ ID NO: 23).

9. The rAAV capsid according to claim 1, wherein the binding moiety comprises or consists of an amino acid sequence selected from SEQ ID NO: 1-147,
preferably selected from SEQ ID NO: 1-26, more preferably selected from SEQ ID NO: 1-3 and 16.

10. The rAAV capsid according to claim 1, wherein the rAAV capsid comprises an expression cassette flanked by at least one AAV inverted terminal repeat (ITR), wherein the expression cassette comprises a nucleic acid molecule encoding at least one gene product.

11. A polynucleotide encoding the recombinant adeno-associated virus (rAAV) capsid according to claim 1.

12. A host cell comprising a polynucleotide according to claim 11, preferably wherein the polynucleotide is an expression vector for expression of the rAAV capsid.

13. A composition comprising the rAAV capsid according to claim 1, preferably wherein the composition is a pharmaceutical composition further comprising at least one pharmaceutically acceptable carrier.

14.-16. (canceled)

17. A method for treating a condition of the central nervous system, the method comprising administering the rAAV capsid according to claim 1 to a subject.

18. The method according to claim 17, wherein the administration is systemically.

19. The method according to claim 18, wherein the administration is intravenously or intraarterially.


SEQUENCE LISTING (308 sequences)

SEQ ID NO: 1 - protein, 9 aa, synthetic construct
  source 1..9: note = CapX-230
AQMHRLQTQ

SEQ ID NO: 2 - protein, 9 aa, synthetic construct
  source 1..9: note = CapX-231
AQYTRLHLT

SEQ ID NO: 3 - protein, 9 aa, synthetic construct
  source 1..9: note = CapX-232
AQRTLSVYP

SEQ ID NO: 4 - protein, 9 aa, synthetic construct
  source 1..9: note = CapX-233
AQLVRPPRS

SEQ ID NO: 5 - protein, 9 aa, synthetic construct
  source 1..9: note = CapX-234
AQTPLRIRG

SEQ ID NO: 6 - protein, 9 aa, synthetic construct
  source 1..9: note = CapX-235
AQRVMSRLP

SEQ ID NO: 7 - protein, 9 aa, synthetic construct
  source 1..9: note = CapX-236
AQSKFRSLL

SEQ ID NO: 8 - protein, 9 aa, synthetic construct
  source 1..9: note = CapX-237
AQPKQRQHA

SEQ ID NO: 9 - protein, 9 aa, synthetic construct
  source 1..9: note = CapX-238
AQASKFRLS

SEQ ID NO: 10 - protein, 9 aa, synthetic construct
  source 1..9: note = CapX-239
AQIAYTTRA

SEQ ID NO: 11 - protein, 9 aa, synthetic construct
  source 1..9: note = CapX-240
AQLMRTVRP

SEQ ID NO: 12 - protein, 9 aa, synthetic construct
  source 1..9: note = CapX-241
AQTWVRSHP

SEQ ID NO: 13 - protein, 9 aa, synthetic construct
  source 1..9: note = CapX-242
AQLGHPWKA

SEQ ID NO: 14 - protein, 9 aa, synthetic construct
  source 1..9: note = CapX-243
AQIVRANLA

SEQ ID NO: 15 - protein, 9 aa, synthetic construct
  source 1..9: note = CapX-244
AQLRVRVSA

SEQ ID NO: 16 - protein, 9 aa, synthetic construct
  source 1..9: note = CapX-245
AQLHRLTVG

SEQ ID NO: 17 - protein, 9 aa, synthetic construct
  source 1..9: note = CapX-246
AQKRGQQFS

SEQ ID NO: 18 - protein, 9 aa, synthetic construct
  source 1..9: note = CapX-247
AQLNSYYGS

SEQ ID NO: 19 - protein, 9 aa, synthetic construct
  source 1..9: note = CapX-248
AQRRLSALG

SEQ ID NO: 20 - protein, 9 aa, synthetic construct
  source 1..9: note = CapX-249
AQLAARRNP

SEQ ID NO: 21 - protein, 9 aa, synthetic construct
  source 1..9: note = CapX-250
AQLVRTKFG

SEQ ID NO: 22 - protein, 9 aa, synthetic construct
  source 1..9: note = CapX-251
AQRPGLARS

SEQ ID NO: 23 - protein, 9 aa, synthetic construct
  source 1..9: note = CapX-252
DGPVPWRVR

SEQ ID NO: 24 - protein, 9 aa, synthetic construct
  source 1..9: note = CapX-253
AQRERLLRG

SEQ ID NO: 25 - protein, 9 aa, synthetic construct
  source 1..9: note = CapX-254
AQFNTLRRL

SEQ ID NO: 26 - protein, 9 aa, synthetic construct
  source 1..9: note = CapX-255
AQTRTSLRQ

SEQ ID NO: 27 - protein, 9 aa, synthetic construct
  source 1..9: note = hCD71_candidate#27
AQSRRATLG

SEQ ID NO: 28 - protein, 9 aa, synthetic construct
  source 1..9: note = hCD71_candidate#28
AQPRLQRPG

SEQ ID NO: 29 - protein, 9 aa, synthetic construct
  source 1..9: note = hCD71_candidate#29
AQRLHLQRR

SEQ ID NO: 30 - protein, 9 aa, synthetic construct
  source 1..9: note = hCD71_candidate#30
AQLPHVRLP

SEQ ID NO: 31 - protein, 9 aa, synthetic construct
  source 1..9: note = hCD71_candidate#31
AQLMMRPRS

SEQ ID NO: 32 - protein, 9 aa, synthetic construct
  source 1..9: note = hCD71_candidate#32
AQRLNRSST

SEQ ID NO: 33 - protein, 9 aa, synthetic construct
  source 1..9: note = hCD71_candidate#33
AQKRSLVNS

SEQ ID NO: 34 - protein, 9 aa, synthetic construct
  source 1..9: note = hCD71_candidate#34
AQPRTHPRM

SEQ ID NO: 35 - protein, 9 aa, synthetic construct
  source 1..9: note = hCD71_candidate#35
AQLWSPRVS

SEQ ID NO: 36 - protein, 9 aa, synthetic construct
  source 1..9: note = hCD71_candidate#36
AQLGRSGRP

SEQ ID NO: 37 - protein, 9 aa, synthetic construct
  source 1..9: note = hCD71_candidate#37
AQRFTIRPA

SEQ ID NO: 38 - protein, 9 aa, synthetic construct
  source 1..9: note = hCD71_candidate#38
AQVPNWKRV

SEQ ID NO: 39 - protein, 9 aa, synthetic construct
  source 1..9: note = hCD71_candidate#39
AQSRQTYHG

SEQ ID NO: 40 - protein, 9 aa, synthetic construct
  source 1..9: note = hCD71_candidate#40
AQKYYQSRD

SEQ ID NO: 41 - protein, 9 aa, synthetic construct
  source 1..9: note = hCD71_candidate#41
AQKARRLSQ

SEQ ID NO: 42 - protein, 9 aa, synthetic construct
  source 1..9: note = hCD71_candidate#42
AQYSRLNAS

SEQ ID NO: 43 - protein, 9 aa, synthetic construct
  source 1..9: note = hCD71_candidate#43
AQRFPFSSA

SEQ ID NO: 44 - protein, 9 aa, synthetic construct
  source 1..9: note = hCD71_candidate#44
AQRGTRGIR

SEQ ID NO: 45 - protein, 9 aa, synthetic construct
  source 1..9: note = hCD71_candidate#45
AQFFRVRVP

SEQ ID NO: 46 - protein, 9 aa, synthetic construct
  source 1..9: note = hCD71_candidate#46
AQRLSRART

SEQ ID NO: 47 - protein, 9 aa, synthetic construct
  source 1..9: note = hCD71_candidate#47
AQIPRIRNA

SEQ ID NO: 48 - protein, 9 aa, synthetic construct
  source 1..9: note = hCD71_candidate#48
AQAPRLLRP

SEQ ID NO: 49 - protein, 9 aa, synthetic construct
  source 1..9: note = hCD71_candidate#49
AQSTIRKAM

SEQ ID NO: 50 - protein, 9 aa, synthetic construct
  source 1..9: note = hCD71_candidate#50
AQCSPVALE

SEQ ID NO: 51 - protein, 9 aa, synthetic construct
  source 1..9: note = hCD71_candidate#51
AQLFDHWLQ

SEQ ID NO: 52 - protein, 9 aa, synthetic construct
  source 1..9: note = hCD71_candidate#52
AQTNHVRFT

SEQ ID NO: 53 - protein, 9 aa, synthetic construct
  source 1..9: note = hCD71_candidate#53
AQSFLWRMA

SEQ ID NO: 54 - protein, 9 aa, synthetic construct
  source 1..9: note = hCD71_candidate#54
DGPASFRQR

SEQ ID NO: 55 - protein, 9 aa, synthetic construct
  source 1..9: note = hCD71_candidate#55
AQLKFPSRS

SEQ ID NO: 56 - protein, 9 aa, synthetic construct
  source 1..9: note = hCD71_candidate#56
AQLPLGRRA

SEQ ID NO: 57 - protein, 9 aa, synthetic construct
  source 1..9: note = hCD71_candidate#57
AQYRARVSA

SEQ ID NO: 58 - protein, 9 aa, synthetic construct
  source 1..9: note = hCD71_candidate#58
AQAAHFRPA

SEQ ID NO: 59 - protein, 9 aa, synthetic construct
  source 1..9: note = hCD71_candidate#59
AQFPPRQSR

SEQ ID NO: 60 - protein, 9 aa, synthetic construct
  source 1..9: note = hCD71_candidate#60
AQHLGLRGP

SEQ ID NO: 61 - protein, 9 aa, synthetic construct
  source 1..9: note = hCD71_candidate#61
AQRRPQALP

SEQ ID NO: 62 - protein, 9 aa, synthetic construct
  source 1..9: note = hCD71_candidate#62
DGPYRGLNR

SEQ ID NO: 63 - protein, 9 aa, synthetic construct
  source 1..9: note = hCD71_candidate#63
AQVIRSRPG

SEQ ID NO: 64 - protein, 9 aa, synthetic construct
  source 1..9: note = hCD71_candidate#64
AQLRAMRMS

SEQ ID NO: 65 - protein, 9 aa, synthetic construct
  source 1..9: note = hCD71_candidate#65
AQLRFRPES

SEQ ID NO: 66 - protein, 9 aa, synthetic construct
  source 1..9: note = hCD71_candidate#66
AQRTPLSRS

SEQ ID NO: 67 - protein, 9 aa, synthetic construct
  source 1..9: note = hCD71_candidate#67
DGPTYRYLR

SEQ ID NO: 68 - protein, 9 aa, synthetic construct
  source 1..9: note = hCD71_candidate#68
AQGLRVTRA

SEQ ID NO: 69 - protein, 9 aa, synthetic construct
  source 1..9: note = hCD71_candidate#69
AQKVIRMGG

SEQ ID NO: 70 - protein, 9 aa, synthetic construct
  source 1..9: note = hCD71_candidate#70
DGPPLLRTR

SEQ ID NO: 71 - protein, 9 aa, synthetic construct
  source 1..9: note = hCD71_candidate#71
AQRFSALVA

SEQ ID NO: 72 - protein, 9 aa, synthetic construct
  source 1..9: note = hCD71_candidate#72
AQYRSLTLR

SEQ ID NO: 73 - protein, 9 aa, synthetic construct
  source 1..9: note = hCD71_candidate#73
AQLRSRWAE

SEQ ID NO: 74 - protein, 9 aa, synthetic construct
  source 1..9: note = hCD71_candidate#74
AQVAAVFKP

SEQ ID NO: 75 - protein, 9 aa, synthetic construct
  source 1..9: note = hCD71_candidate#75
AQLLSLRRP

SEQ ID NO: 76 - protein, 9 aa, synthetic construct
  source 1..9: note = hCD71_candidate#76
AQQVSRSLY

SEQ ID NO: 77 - protein, 9 aa, synthetic construct
  source 1..9: note = hCD71_candidate#77
AQRARPYQP

SEQ ID NO: 78 - protein, 9 aa, synthetic construct
  source 1..9: note = hCD71_candidate#78
AQRFARVPS

SEQ ID NO: 79 - protein, 9 aa, synthetic construct
  source 1..9: note = hCD71_candidate#79
AQGAYHRLQ

SEQ ID NO: 80 - protein, 9 aa, synthetic construct
  source 1..9: note = hCD71_candidate#80
AQMARFPKP

SEQ ID NO: 81 - protein, 9 aa, synthetic construct
  source 1..9: note = hCD71_candidate#81
AQFRHLAGG

SEQ ID NO: 82 - protein, 9 aa, synthetic construct
  source 1..9: note = hCD71_candidate#82
AQLARTTVP

SEQ ID NO: 83 - protein, 9 aa, synthetic construct
  source 1..9: note = hCD71_candidate#83
AQLKVRLSG

SEQ ID NO: 84 - protein, 9 aa, synthetic construct
  source 1..9: note = hCD71_candidate#84
AQSARFVVV

SEQ ID NO: 85 - protein, 9 aa, synthetic construct
  source 1..9: note = hCD71_candidate#85
AQGPGRRGP

SEQ ID NO: 86 - protein, 9 aa, synthetic construct
  source 1..9: note = hCD71_candidate#86
AQKRPSVTA

SEQ ID NO: 87 - protein, 9 aa, synthetic construct
  source 1..9: note = hCD71_candidate#87
AQRNLTMRA

SEQ ID NO: 88 - protein, 9 aa, synthetic construct
  source 1..9: note = hCD71_candidate#88
AQLGAFSRA

SEQ ID NO: 89 - protein, 9 aa, synthetic construct
  source 1..9: note = hCD71_candidate#89
AQLRSRNNP

SEQ ID NO: 90 - protein, 9 aa, synthetic construct
  source 1..9: note = hCD71_candidate#90
AQVLSVSRV

SEQ ID NO: 91 - protein, 9 aa, synthetic construct
  source 1..9: note = hCD71_candidate#91
AQRNPRPTS

SEQ ID NO: 92 - protein, 9 aa, synthetic construct
  source 1..9: note = hCD71_candidate#92
AQMRTKLLS

SEQ ID NO: 93 - protein, 9 aa, synthetic construct
  source 1..9: note = hCD71_candidate#93
AQLLLRQRP

SEQ ID NO: 94 - protein, 9 aa, synthetic construct
  source 1..9: note = hCD71_candidate#94
AQGLYKAPR

SEQ ID NO: 95 - protein, 9 aa, synthetic construct
  source 1..9: note = hCD71_candidate#95
AQRTVSKML

SEQ ID NO: 96 - protein, 9 aa, synthetic construct
  source 1..9: note = hCD71_candidate#96
AQKVPARYN

SEQ ID NO: 97 - protein, 9 aa, synthetic construct
  source 1..9: note = hCD71_candidate#97
AQRVPFVRV

SEQ ID NO: 98 - protein, 9 aa, synthetic construct
  source 1..9: note = hCD71_candidate#98
AQPRPPTRS

SEQ ID NO: 99 - protein, 9 aa, synthetic construct
  source 1..9: note = hCD71_candidate#99
AQLYMLGRP

SEQ ID NO: 100 - protein, 9 aa, synthetic construct
  source 1..9: note = hCD71_candidate#100
AQLRAFAKL

SEQ ID NO: 101 - protein, 9 aa, synthetic construct
  source 1..9: note = hCD71_candidate#101
AQRSPLLRP

SEQ ID NO: 102 - protein, 9 aa, synthetic construct
  source 1..9: note = hCD71_candidate#102
AQRRSPAAL

SEQ ID NO: 103 - protein, 9 aa, synthetic construct
  source 1..9: note = hCD71_candidate#103
AQIRRVDIA

SEQ ID NO: 104 - protein, 9 aa, synthetic construct
  source 1..9: note = hCD71_candidate#104
AQGVPMRRP

SEQ ID NO: 105 - protein, 9 aa, synthetic construct
  source 1..9: note = hCD71_candidate#105
AQYRVAQKF

SEQ ID NO: 106 - protein, 9 aa, synthetic construct
  source 1..9: note = hCD71_candidate#106
AQSRLLRQN

SEQ ID NO: 107 - protein, 9 aa, synthetic construct
  source 1..9: note = hCD71_candidate#107
AQRFSSRLA

SEQ ID NO: 108 - protein, 9 aa, synthetic construct
  source 1..9: note = hCD71_candidate#108
AQATTSRVR

SEQ ID NO: 109 - protein, 9 aa, synthetic construct
  source 1..9: note = hCD71_candidate#109
AQAALHKWM

SEQ ID NO: 110 - protein, 9 aa, synthetic construct
  source 1..9: note = hCD71_candidate#110
DGPAQWHRR

SEQ ID NO: 111 - protein, 9 aa, synthetic construct
  source 1..9: note = hCD71_candidate#111
AQRLAVRQA

SEQ ID NO: 112 - protein, 9 aa, synthetic construct
  source 1..9: note = hCD71_candidate#112
AQARQLGRP

SEQ ID NO: 113 - protein, 9 aa, synthetic construct
  source 1..9: note = hCD71_candidate#113
AQRPLMRSA

SEQ ID NO: 114 - protein, 9 aa, synthetic construct
  source 1..9: note = hCD71_candidate#114
DGPSSIRSR

SEQ ID NO: 115 - protein, 9 aa, synthetic construct
  source 1..9: note = hCD71_candidate#115
AQFQRIHMM

SEQ ID NO: 116 - protein, 9 aa, synthetic construct
  source 1..9: note = hCD71_candidate#116
AQRRALGSA

SEQ ID NO: 117 - protein, 9 aa, synthetic construct
  source 1..9: note = hCD71_candidate#117
AQLLPRRPR

SEQ ID NO: 118 - protein, 9 aa, synthetic construct
  source 1..9: note = hCD71_candidate#118
AQRWAASLN

SEQ ID NO: 119 - protein, 9 aa, synthetic construct
  source 1..9: note = hCD71_candidate#119
AQCKSYQLA

SEQ ID NO: 120 - protein, 9 aa, synthetic construct
  source 1..9: note = hCD71_candidate#120
AQRAAGYRG

SEQ ID NO: 121 - protein, 9 aa, synthetic construct
  source 1..9: note = hCD71_candidate#121
AQLWARQGG

SEQ ID NO: 122 - protein, 9 aa, synthetic construct
  source 1..9: note = hCD71_candidate#122
AQPLRASRP

SEQ ID NO: 123 - protein, 9 aa, synthetic construct
  source 1..9: note = hCD71_candidate#123
AQKLPYRLG

SEQ ID NO: 124 - protein, 9 aa, synthetic construct
  source 1..9: note = hCD71_candidate#124
DGPYYGGRR

SEQ ID NO: 125 - protein, 9 aa, synthetic construct
  source 1..9: note = hCD71_candidate#125
AQPRIKHGF

SEQ ID NO: 126 - protein, 9 aa, synthetic construct
  source 1..9: note = hCD71_candidate#126
AQSLSRLRP

SEQ ID NO: 127 - protein, 9 aa, synthetic construct
  source 1..9: note = hCD71_candidate#127
AQAVRLVSR

SEQ ID NO: 128 - protein, 9 aa, synthetic construct
  source 1..9: note = hCD71_candidate#128
AQTQPPRKY

SEQ ID NO: 129 - protein, 9 aa, synthetic construct
  source 1..9: note = hCD71_candidate#129
AQLWRGGGQ

SEQ ID NO: 130 - protein, 9 aa, synthetic construct
  source 1..9: note = hCD71_candidate#130
AQRTAFRVP

SEQ ID NO: 131 - protein, 9 aa, synthetic construct
  source 1..9: note = hCD71_candidate#131
AQRAGRPYY

SEQ ID NO: 132 - protein, 9 aa, synthetic construct
  source 1..9: note = hCD71_candidate#132
AQQTSHRVV

SEQ ID NO: 133 - protein, 9 aa, synthetic construct
  source 1..9: note = hCD71_candidate#133
AQQWKIGGL

SEQ ID NO: 134 - protein, 9 aa, synthetic construct
  source 1..9: note = hCD71_candidate#134
AQRKPAASG

SEQ ID NO: 135 - protein, 9 aa, synthetic construct
  source 1..9: note = hCD71_candidate#135
AQTATYSRS

SEQ ID NO: 136 - protein, 9 aa, synthetic construct
  source 1..9: note = hCD71_candidate#136
AQRSAARPV

SEQ ID NO: 137 - protein, 9 aa, synthetic construct
  source 1..9: note = hCD71_candidate#137
AQPGSILAR

SEQ ID NO: 138 - protein, 9 aa, synthetic construct
  source 1..9: note = hCD71_candidate#138
AQQSRLRVP

SEQ ID NO: 139 - protein, 9 aa, synthetic construct
  source 1..9: note = hCD71_candidate#139
AQWWHATRA

SEQ ID NO: 140 - protein, 9 aa, synthetic construct
  source 1..9: note = hCD71_candidate#140
AQGVSRRGS

SEQ ID NO: 141 - protein, 9 aa, synthetic construct
  source 1..9: note = hCD71_candidate#141
AQDPPRGRR

SEQ ID NO: 142 - protein, 9 aa, synthetic construct
  source 1..9: note = hCD71_candidate#142
AQYRQAVHV

SEQ ID NO: 143 - protein, 9 aa, synthetic construct
  source 1..9: note = hCD71_candidate#143
AQSVPTRRP

SEQ ID NO: 144 - protein, 9 aa, synthetic construct
  source 1..9: note = hCD71_candidate#144
AQSRRIEKV

SEQ ID NO: 145 - protein, 9 aa, synthetic construct
  source 1..9: note = hCD71_candidate#145
AQTPMLVRG

SEQ ID NO: 146 - protein, 9 aa, synthetic construct
  source 1..9: note = hCD71_candidate#146
AQPLTAARR

SEQ ID NO: 147 - protein, 9 aa, synthetic construct
  source 1..9: note = hCD71_candidate#147
AQVFHKLRS

SEQ ID NO: 148 - DNA, 27 bp, synthetic construct
  source 1..27: mol_type = other DNA; note = CapX-230
gcccaaatgcatcgtcttcagacgcag

SEQ ID NO: 149 - DNA, 27 bp, synthetic construct
  source 1..27: mol_type = other DNA; note = CapX-231
gcccaatatactcggttgcatctgacg

SEQ ID NO: 150 - DNA, 27 bp, synthetic construct
  source 1..27: mol_type = other DNA; note = CapX-232
gcccaacggacgctttctgtgtatccg

SEQ ID NO: 151 - DNA, 27 bp, synthetic construct
  source 1..27: mol_type = other DNA; note = CapX-233
gcccaattggttcgtcctccgcggtcg

SEQ ID NO: 152 - DNA, 27 bp, synthetic construct
  source 1..27: mol_type = other DNA; note = CapX-234
gcccaaactccgttgcggattcggggt

SEQ ID NO: 153 - DNA, 27 bp, synthetic construct
  source 1..27: mol_type = other DNA; note = CapX-235
gcccaacgtgtgatgtcgcgtcttcct

SEQ ID NO: 154 - DNA, 27 bp, synthetic construct
  source 1..27: mol_type = other DNA; note = CapX-236
gcccaatctaagtttcgtagtttgttg

SEQ ID NO: 155 - DNA, 27 bp, synthetic construct
  source 1..27: mol_type = other DNA; note = CapX-237
gcccaacctaagcagcggcagcatgct

SEQ ID NO: 156 - DNA, 27 bp, synthetic construct
  source 1..27: mol_type = other DNA; note = CapX-238
gcccaagcgtctaagtttcggttgagt

SEQ ID NO: 157 - DNA, 27 bp, synthetic construct
  source 1..27: mol_type = other DNA; note = CapX-239
gcccaaattgcttatactacgcgtgcg

SEQ ID NO: 158 - DNA, 27 bp, synthetic construct
  source 1..27: mol_type = other DNA; note = CapX-240
gcccaacttatgcggacggttcgtccg

SEQ ID NO: 159 - DNA, 27 bp, synthetic construct
  source 1..27: mol_type = other DNA; note = CapX-241
gcccaaacgtgggtgcggtcgcatccg

SEQ ID NO: 160 - DNA, 27 bp, synthetic construct
  source 1..27: mol_type = other DNA; note = CapX-242
gcccaactgggtcatccttggaaggct

SEQ ID NO: 161 - DNA, 27 bp, synthetic construct
  source 1..27: mol_type = other DNA; note = CapX-243
gcccaaattgttcgggcgaatttggcg

SEQ ID NO: 162 - DNA, 27 bp, synthetic construct
  source 1..27: mol_type = other DNA; note = CapX-244
gcccaattgcgtgtgagggtgtcggcg

SEQ ID NO: 163 - DNA, 27 bp, synthetic construct
  source 1..27: mol_type = other DNA; note = CapX-245
gcccaacttcatcggctgacggtgggg

SEQ ID NO: 164 - DNA, 27 bp, synthetic construct
  source 1..27: mol_type = other DNA; note = CapX-246
gcccaaaagagggggcagcagttttcg

SEQ ID NO: 165 - DNA, 27 bp, synthetic construct
  source 1..27: mol_type = other DNA; note = CapX-247
gcccaattgaattcttattatggttct

SEQ ID NO: 166 - DNA, 27 bp, synthetic construct
  source 1..27: mol_type = other DNA; note = CapX-248
gcccaacggcggctttcggcgcttggt

SEQ ID NO: 167 - DNA, 27 bp, synthetic construct
  source 1..27: mol_type = other DNA; note = CapX-249
gcccaattggcggcgcgtcgtaatccg

SEQ ID NO: 168 - DNA, 27 bp, synthetic construct
  source 1..27: mol_type = other DNA; note = CapX-250
gcccaattggtgcgtactaagtttggg

SEQ ID NO: 169 - DNA, 27 bp, synthetic construct
  source 1..27: mol_type = other DNA; note = CapX-251
gcccaacgtcctggtcttgctcggtcg

SEQ ID NO: 170 - DNA, 27 bp, synthetic construct
  source 1..27: mol_type = other DNA; note = CapX-252
gacggacctgttccttggcgtgttcgt

SEQ ID NO: 171 - DNA, 27 bp, synthetic construct
  source 1..27: mol_type = other DNA; note = CapX-253
gcccaacgtgagcggttgcttcggggg

SEQ ID NO: 172 - DNA, 27 bp, synthetic construct
  source 1..27: mol_type = other DNA; note = CapX-254
gcccaatttaatacgttgcggcggttg

SEQ ID NO: 173 - DNA, 27 bp, synthetic construct
  source 1..27: mol_type = other DNA; note = CapX-255
gcccaaacgcgtacgtcgttgcgtcag

SEQ ID NO: 174 - DNA, 27 bp, synthetic construct
  source 1..27: mol_type = other DNA; note = hCD71_candidate#27
gcccaatctcggcgtgcgacgcttggg

SEQ ID NO: 175 - DNA, 27 bp, synthetic construct
  source 1..27: mol_type = other DNA; note = hCD71_candidate#28
gcccaacctaggctgcagaggccgggg

SEQ ID NO: 176 - DNA, 27 bp, synthetic construct
  source 1..27: mol_type = other DNA; note = hCD71_candidate#29
gcccaacggttgcatttgcagcggagg

SEQ ID NO: 177 - DNA, 27 bp, synthetic construct
  source 1..27: mol_type = other DNA; note = hCD71_candidate#30
gcccaattgccgcatgtgcgtctgcct

SEQ ID NO: 178 - DNA, 27 bp, synthetic construct
  source 1..27: mol_type = other DNA; note = hCD71_candidate#31
gcccaattgatgatgcgtccgaggtct

SEQ ID NO: 179 - DNA, 27 bp, synthetic construct
  source 1..27: mol_type = other DNA; note = hCD71_candidate#32
gcccaacggctgaatcgttcgtcgacg

SEQ ID NO: 180 - DNA, 27 bp, synthetic construct
  source 1..27: mol_type = other DNA; note = hCD71_candidate#33
gcccaaaagcggtcgcttgttaattct

SEQ ID NO: 181 - DNA, 27 bp, synthetic construct
  source 1..27: mol_type = other DNA; note = hCD71_candidate#34
gcccaaccgcggactcatcctcgtatg

SEQ ID NO: 182 - DNA, 27 bp, synthetic construct
  source 1..27: mol_type = other DNA; note = hCD71_candidate#35
gcccaattgtggtctcctcgggtttcg

SEQ ID NO: 183 - DNA, 27 bp, synthetic construct
  source 1..27: mol_type = other DNA; note = hCD71_candidate#36
gcccaacttggtaggtctgggaggccg

SEQ ID NO: 184 - DNA, 27 bp, synthetic construct
  source 1..27: mol_type = other DNA; note = hCD71_candidate#37
gcccaaaggtttacgattcgtcctgcg

SEQ ID NO: 185 - DNA, 27 bp, synthetic construct
  source 1..27: mol_type = other DNA; note = hCD71_candidate#38
gcccaagtgcctaattggaagcgggtt

SEQ ID NO: 186 - DNA, 27 bp, synthetic construct
  source 1..27: mol_type = other DNA; note = hCD71_candidate#39
gcccaatcgaggcagacgtatcatggt

SEQ ID NO: 187 - DNA, 27 bp, synthetic construct
  source 1..27: mol_type = other DNA; note = hCD71_candidate#40
gcccaaaagtattatcagagtcgtgat

SEQ ID NO: 188 - DNA, 27 bp, synthetic construct
  source 1..27: mol_type = other DNA; note = hCD71_candidate#41
gcccaaaaggcgaggcggctttctcag

SEQ ID NO: 189 - DNA, 27 bp, synthetic construct
  source 1..27: mol_type = other DNA; note = hCD71_candidate#42
gcccaatatagtcggctgaatgcgtcg

SEQ ID NO: 190 - DNA, 27 bp, synthetic construct
  source 1..27: mol_type = other DNA; note = hCD71_candidate#43
gcccaacggtttccgttttcttcggcg

SEQ ID NO: 191 - DNA, 27 bp, synthetic construct
  source 1..27: mol_type = other DNA; note = hCD71_candidate#44
gcccaacgggggacgcggggtattcgg

SEQ ID NO: 192 - DNA, 27 bp, synthetic construct
  source 1..27: mol_type = other DNA; note = hCD71_candidate#45
gcccaattttttcgtgtgagggttccg

SEQ ID NO: 193 - DNA, 27 bp, synthetic construct
  source 1..27: mol_type = other DNA; note = hCD71_candidate#46
gcccaacggttgtctcgtgctcggact

SEQ ID NO: 194 - DNA, 27 bp, synthetic construct
  source 1..27: mol_type = other DNA; note = hCD71_candidate#47
gcccaaattcctcggattaggaatgcg

SEQ ID NO: 195 - DNA, 27 bp, synthetic construct
  source 1..27: mol_type = other DNA; note = hCD71_candidate#48
gcccaagcgccgcgtctgctgcgtcct

SEQ ID NO: 196 - DNA, 27 bp, synthetic construct
  source 1..27: mol_type = other DNA; note = hCD71_candidate#49
gcccaatctacgattaggaaggcgatg

SEQ ID NO: 197 - DNA, 27 bp, synthetic construct
  source 1..27: mol_type = other DNA; note = hCD71_candidate#50
gcccaatgttctcctgtggcgctggag

SEQ ID NO: 198 - DNA, 27 bp, synthetic construct
  source 1..27: mol_type = other DNA; note = hCD71_candidate#51
gcccaactttttgatcattggcttcag

SEQ ID NO: 199 - DNA, 27 bp, synthetic construct
  source 1..27: mol_type = other DNA; note = hCD71_candidate#52
gcccaaactaatcatgttcgttttacg

SEQ ID NO: 200 - DNA, 27 bp, synthetic construct
  source 1..27: mol_type = other DNA; note = hCD71_candidate#53
gcccaaagttttctttggcgtatggct

SEQ ID NO: 201 - DNA, 27 bp, synthetic construct
  source 1..27: mol_type = other DNA; note = hCD71_candidate#54
gacggacctgctagttttcgtcagcgt

SEQ ID NO: 202 - DNA, 27 bp, synthetic construct
  source 1..27: mol_type = other DNA; note = hCD71_candidate#55
gcccaattgaagtttccgagtcgttcg

SEQ ID NO: 203 - DNA, 27 bp, synthetic construct
  source 1..27: mol_type = other DNA; note = hCD71_candidate#56
gcccaactgcctcttggtaggcgggct

SEQ ID NO: 204 - DNA, 27 bp, synthetic construct
  source 1..27: mol_type = other DNA; note = hCD71_candidate#57
gcccaatatcgtgcgcgggttagtgct

SEQ ID NO: 205 - DNA, 27 bp, synthetic construct
  source 1..27: mol_type = other DNA; note = hCD71_candidate#58
gcccaagctgctcattttcgtccggct

SEQ ID NO: 206 - DNA, 27 bp, synthetic construct
  source 1..27: mol_type = other DNA; note = hCD71_candidate#59
gcccaatttcctcctaggcagagtcgg

SEQ ID NO: 207 - DNA, 27 bp, synthetic construct
  source 1..27: mol_type = other DNA; note = hCD71_candidate#60
gcccaacatttgggtttgcgtggtcct

SEQ ID NO: 208 - DNA, 27 bp, synthetic construct
  source 1..27: mol_type = other DNA; note = hCD71_candidate#61
gcccaacggcggccgcaggcgcttcct

SEQ ID NO: 209 - DNA, 27 bp, synthetic construct
  source 1..27: mol_type = other DNA; note = hCD71_candidate#62
gacggaccttatagggggcttaatcgt

SEQ ID NO: 210 - DNA, 27 bp, synthetic construct
  source 1..27: mol_type = other DNA; note = hCD71_candidate#63
gcccaagtgattcgttctcggcctggt

SEQ ID NO: 211 - DNA, 27 bp, synthetic construct
  source 1..27: mol_type = other DNA; note = hCD71_candidate#64
gcccaattgagggcgatgcggatgtct

SEQ ID NO: 212 - DNA, 27 bp, synthetic construct
  source 1..27: mol_type = other DNA; note = hCD71_candidate#65
gcccaattgaggtttaggccggagtcg

SEQ ID NO: 213 - DNA, 27 bp, synthetic construct
  source 1..27: mol_type = other DNA; note = hCD71_candidate#66
gcccaacgtacgcctctttctcgtagt

SEQ ID NO: 214 - DNA, 27 bp, synthetic construct
  source 1..27: mol_type = other DNA; note = hCD71_candidate#67
gacggacctacgtatcggtatcttcgt

SEQ ID NO: 215 - DNA, 27 bp, synthetic construct
  source 1..27: mol_type = other DNA; note = hCD71_candidate#68
gcccaagggcttcgtgttacgcgggcg

SEQ ID NO: 216 - DNA, 27 bp, synthetic construct
  source 1..27: mol_type = other DNA; note = hCD71_candidate#69
gcccaaaaggttattcggatgggtggt

SEQ ID NO: 217 - DNA, 27 bp, synthetic construct
  source 1..27: mol_type = other DNA; note = hCD71_candidate#70
gacggacctcctctgctgcggactcgt

SEQ ID NO: 218 - DNA, 27 bp, synthetic construct
  source 1..27: mol_type = other DNA; note = hCD71_candidate#71
gcccaacggttttcggcgttggttgcg

SEQ ID NO: 219 - DNA, 27 bp, synthetic construct
  source 1..27: mol_type = other DNA; note = hCD71_candidate#72
gcccaatatcggtctttgactcttcgg

SEQ ID NO: 220 - DNA, 27 bp, synthetic construct
  source 1..27: mol_type = other DNA; note = hCD71_candidate#73
gcccaacttcgttctcggtgggctgag

SEQ ID NO: 221 - DNA, 27 bp, synthetic construct
  source 1..27: mol_type = other DNA; note = hCD71_candidate#74
gcccaagtggctgcggtttttaagcct

SEQ ID NO: 222 - DNA, 27 bp, synthetic construct
  source 1..27: mol_type = other DNA; note = hCD71_candidate#75
gcccaacttctgtcgcttcggcgtcct

SEQ ID NO: 223 - DNA, 27 bp, synthetic construct
  source 1..27: mol_type = other DNA; note = hCD71_candidate#76
gcccaacaggttagtaggtctctttat

SEQ ID NO: 224 - DNA, 27 bp, synthetic construct
  source 1..27: mol_type = other DNA; note = hCD71_candidate#77
gcccaacgggcgcgtccgtatcagccg

SEQ ID NO: 225 - DNA, 27 bp, synthetic construct
  source 1..27: mol_type = other DNA; note = hCD71_candidate#78
gcccaacggtttgctcgtgttccgtcg

SEQ ID NO: 226 - DNA, 27 bp, synthetic construct
  source 1..27: mol_type = other DNA; note = hCD71_candidate#79
gcccaaggtgcttatcatcggctgcag

SEQ ID NO: 227 - DNA, 27 bp, synthetic construct
  source 1..27: mol_type = other DNA; note = hCD71_candidate#80
gcccaaatggcgcggtttccgaagccg

SEQ ID NO: 228 - DNA, 27 bp, synthetic construct
  source 1..27: mol_type = other DNA; note = hCD71_candidate#81
gcccaatttcgtcatctggcggggggt

SEQ ID NO: 229 - DNA, 27 bp, synthetic construct
  source 1..27: mol_type = other DNA; note = hCD71_candidate#82
gcccaattggctcgtacgacggttccg

SEQ ID NO: 230 - DNA, 27 bp, synthetic construct
  source 1..27: mol_type = other DNA; note = hCD71_candidate#83
gcccaactgaaggttcgtctgtctggt

SEQ ID NO: 231 - DNA, 27 bp, synthetic construct
  source 1..27: mol_type = other DNA; note = hCD71_candidate#84
gcccaatcggcgaggtttgttgttgtg

SEQ ID NO: 232 - DNA, 27 bp, synthetic construct
  source 1..27: mol_type = other DNA; note = hCD71_candidate#85
gcccaagggccgggtcgtcgggggcct

SEQ ID NO: 233 - DNA, 27 bp, synthetic construct
  source 1..27: mol_type = other DNA; note = hCD71_candidate#86
gcccaaaagcggccttcggttactgcg

SEQ ID NO: 234 - DNA, 27 bp, synthetic construct
  source 1..27: mol_type = other DNA; note = hCD71_candidate#87
gcccaaaggaatcttactatgcgggcg

SEQ ID NO: 235 - DNA, 27 bp, synthetic construct
  source 1..27: mol_type = other DNA; note = hCD71_candidate#88
gcccaattgggggctttttctcgggct

SEQ ID NO: 236 - DNA, 27 bp, synthetic construct
  source 1..27: mol_type = other DNA; note = hCD71_candidate#89
gcccaacttcgttcgcggaataatccg

SEQ ID NO: 237 - DNA, 27 bp, synthetic construct
  source 1..27: mol_type = other DNA; note = hCD71_candidate#90
gcccaagtgttgtcggtttcgagggtg

SEQ ID NO: 238 - DNA, 27 bp, synthetic construct
  source 1..27: mol_type = other DNA; note = hCD71_candidate#91
gcccaacggaatcctcggccgactagt

SEQ ID NO: 239 - DNA, 27 bp, synthetic construct
  source 1..27: mol_type = other DNA; note = hCD71_candidate#92
gcccaaatgcggacgaagcttctttct

SEQ ID NO: 240 - DNA, 27 bp, synthetic construct
  source 1..27: mol_type = other DNA; note = hCD71_candidate#93
gcccaattgcttttgcgtcagcggcct

SEQ ID NO: 241 - DNA, 27 bp, synthetic construct
  source 1..27: mol_type = other DNA; note = hCD71_candidate#94
gcccaaggtctttataaggcgcctcgt

SEQ ID NO: 242 - DNA, 27 bp, synthetic construct
  source 1..27: mol_type = other DNA; note = hCD71_candidate#95
gcccaacgtactgtgtcgaagatgctg

SEQ ID NO: 243 - DNA, 27 bp, synthetic construct
  source 1..27: mol_type = other DNA; note = hCD71_candidate#96
gcccaaaaggttccggcgcggtataat

SEQ ID NO: 244 - DNA, 27 bp, synthetic construct
  source 1..27: mol_type = other DNA; note = hCD71_candidate#97
gcccaacgtgtgccgtttgtgagggtg

SEQ ID NO: 245 - DNA, 27 bp, synthetic construct
  source 1..27: mol_type = other DNA; note = hCD71_candidate#98
gcccaaccgcggcctccgactcggtcg

SEQ ID NO: 246 - DNA, 27 bp, synthetic construct
  source 1..27: mol_type = other DNA; note = hCD71_candidate#99
gcccaactttatatgttgggtcggccg

SEQ ID NO: 247 - DNA, 27 bp, synthetic construct
  source 1..27: mol_type = other DNA; note = hCD71_candidate#100
gcccaattgagggcgtttgcgaagttg

SEQ ID NO: 248 - DNA, 27 bp, synthetic construct
  source 1..27: mol_type = other DNA; note = hCD71_candidate#101
gcccaacggtctcctctgcttaggccg

SEQ ID NO: 249 - DNA, 27 bp, synthetic construct
  source 1..27: mol_type = other DNA; note = hCD71_candidate#102
gcccaacggcggtcgcctgcggcgctg

SEQ ID NO: 250 - DNA, 27 bp, synthetic construct
  source 1..27: mol_type = other DNA; note = hCD71_candidate#103
gcccaaattcgtagggttgatattgcg

SEQ ID NO: 251 - DNA, 27 bp, synthetic construct
  source 1..27: mol_type = other DNA; note = hCD71_candidate#104
gcccaaggggtgcctatgcgtcgtcct

SEQ ID NO: 252 - DNA, 27 bp, synthetic construct
  source 1..27: mol_type = other DNA; note = hCD71_candidate#105
gcccaatatcgtgtggctcagaagttt

SEQ ID NO: 253 - DNA, 27 bp, synthetic construct
  source 1..27: mol_type = other DNA; note = hCD71_candidate#106
gcccaaagtcgtctgttgcggcagaat

SEQ ID NO: 254 - DNA, 27 bp, synthetic construct
  source 1..27: mol_type = other DNA; note = hCD71_candidate#107
gcccaacggttttcttcgaggctggcg

SEQ ID NO: 255 - DNA, 27 bp, synthetic construct
  source 1..27: mol_type = other DNA; note = hCD71_candidate#108
gcccaagcgacgacgtctcgggttcgg

SEQ ID NO: 256 - DNA, 27 bp, synthetic construct
  source 1..27: mol_type = other DNA; note = hCD71_candidate#109
gcccaagctgcgcttcataagtggatg

SEQ ID NO: 257 - DNA, 27 bp, synthetic construct
  source 1..27: mol_type = other DNA; note = hCD71_candidate#110
gacggacctgctcagtggcatcgtcgt

SEQ ID NO: 258 - DNA, 27 bp, synthetic construct
  source 1..27: mol_type = other DNA; note = hCD71_candidate#111
gcccaacgtcttgcggttcgtcaggcg

SEQ ID NO: 259 - DNA, 27 bp, synthetic construct
  source 1..27: mol_type = other DNA; note = hCD71_candidate#112
gcccaagcgcggcagctgggtcgtccg

SEQ ID NO: 260 - DNA, 27 bp, synthetic construct
  source 1..27: mol_type = other DNA; note = hCD71_candidate#113
gcccaaaggccgttgatgcgtagtgct

SEQ ID NO: 261 - DNA, 27 bp, synthetic construct
  source 1..27: mol_type = other DNA; note = hCD71_candidate#114
gacggaccttctagtattcgttcgcgt

SEQ ID NO: 262 - DNA, 27 bp, synthetic construct
  source 1..27: mol_type = other DNA; note = hCD71_candidate#115
gcccaatttcagcgtattcatatgatg

SEQ ID NO: 263 - DNA, 27 bp, synthetic construct
  source 1..27: mol_type = other DNA; note = hCD71_candidate#116
gcccaacggcgggctttggggtcggct

SEQ ID NO: 264 - DNA, 27 bp, synthetic construct
  source 1..27: mol_type = other DNA; note = hCD71_candidate#117
gcccaacttcttccgcgtaggccgcgg

SEQ ID NO: 265 - DNA, 27 bp, synthetic construct
  source 1..27: mol_type = other DNA; note = hCD71_candidate#118
gcccaaaggtgggcggcttcgttgaat

SEQ ID NO: 266 - DNA, 27 bp, synthetic construct
  source 1..27: mol_type = other DNA; note = hCD71_candidate#119
gcccaatgtaagtcgtatcagttggct

SEQ ID NO: 267 - DNA, 27 bp, synthetic construct
  source 1..27: mol_type = other DNA; note = hCD71_candidate#120
gcccaacgggcggcggggtataggggt

SEQ ID NO: 268 - DNA, 27 bp, synthetic construct
  source 1..27: mol_type = other DNA; note = hCD71_candidate#121
gcccaactgtgggctcgtcagggtggt

SEQ ID NO: 269 - DNA, 27 bp, synthetic construct
  source 1..27: mol_type = other DNA; note = hCD71_candidate#122
gcccaaccgcttagggcgagtcggccg

SEQ ID NO: 270 - DNA, 27 bp, synthetic construct
  source 1..27: mol_type = other DNA; note = hCD71_candidate#123
gcccaaaagttgccttatcgtttgggt

SEQ ID NO: 271 - DNA, 27 bp, synthetic construct
  source 1..27: mol_type = other DNA; note = hCD71_candidate#124
gacggaccttattatgggggtcgtcgt

SEQ ID NO: 272 - DNA, 27 bp, synthetic construct
  source 1..27: mol_type = other DNA; note = hCD71_candidate#125
gcccaaccgaggattaagcatgggttt

SEQ ID NO: 273 - DNA, 27 bp, synthetic construct
  source 1..27: mol_type = other DNA; note = hCD71_candidate#126
gcccaatcgctgtctcggcttcggccg

SEQ ID NO: 274 - DNA, 27 bp, synthetic construct
  source 1..27: mol_type = other DNA; note = hCD71_candidate#127
gcccaagctgtgcggcttgtgtctcgg

SEQ ID NO: 275 - DNA, 27 bp, synthetic construct
  source 1..27: mol_type = other DNA; note = hCD71_candidate#128
gcccaaactcagcctccgcgtaagtat

SEQ ID NO: 276 - DNA, 27 bp, synthetic construct
  source 1..27: mol_type = other DNA; note = hCD71_candidate#129
gcccaactgtggcgggggggtgggcag

SEQ ID NO: 277 - DNA, 27 bp, synthetic construct
  source 1..27: mol_type = other DNA; note = hCD71_candidate#130
gcccaaaggacggcctttcgggtgccg

SEQ ID NO: 278 - DNA, 27 bp, synthetic construct
  source 1..27: mol_type = other DNA; note = hCD71_candidate#131
gcccaaagggcgggtcgtccttattat

SEQ ID NO: 279 - DNA, 27 bp, synthetic construct
  source 1..27: mol_type = other DNA; note = hCD71_candidate#132
gcccaacagacgtcgcatcgtgtggtt

SEQ ID NO: 280 - DNA, 27 bp, synthetic construct
  source 1..27: mol_type = other DNA; note = hCD71_candidate#133
gcccaacagtggaagattggtgggttg

SEQ ID NO: 281 - DNA, 27 bp, synthetic construct
  source 1..27: mol_type = other DNA; note = hCD71_candidate#134
gcccaacggaagccggctgcttctggt

SEQ ID NO: 282 - DNA, 27 bp, synthetic construct
  source 1..27: mol_type = other DNA; note = hCD71_candidate#135
gcccaaacggcgacttatagtcggtcg

SEQ ID NO: 283 - DNA, 27 bp, synthetic construct
  source 1..27: mol_type = other DNA; note = hCD71_candidate#136
gcccaaaggtcggctgctcggccggtt

SEQ ID NO: 284 - DNA, 27 bp, synthetic construct
  source 1..27: mol_type = other DNA; note = hCD71_candidate#137
gcccaacctgggtctattctggctcgg

SEQ ID NO: 285 - DNA, 27 bp, synthetic construct
  source 1..27: mol_type = other DNA; note = hCD71_candidate#138
gcccaacagtcgcggctgagggtgccg

SEQ ID NO: 286 - DNA, 27 bp, synthetic construct
  source 1..27: mol_type = other DNA; note = hCD71_candidate#139
gcccaatggtggcatgctactcgggcg

SEQ ID NO: 287 - DNA, 27 bp, synthetic construct
  source 1..27: mol_type = other DNA; note = hCD71_candidate#140
gcccaaggggtgtcgcggcgtgggtcg

SEQ ID NO: 288 - DNA, 27 bp, synthetic construct
  source 1..27: mol_type = other DNA; note = hCD71_candidate#141
gcccaagatcctccgcgggggcgtagg

SEQ ID NO: 289 - DNA, 27 bp, synthetic construct
  source 1..27: mol_type = other DNA; note = hCD71_candidate#142
gcccaatatcggcaggctgtgcatgtg

SEQ ID NO: 290 - DNA, 27 bp, synthetic construct
  source 1..27: mol_type = other DNA; note = hCD71_candidate#143
gcccaatcggttcctacgaggcgtcct

SEQ ID NO: 291 - DNA, 27 bp, synthetic construct
  source 1..27: mol_type = other DNA; note = hCD71_candidate#144
gcccaatcgaggcggattgagaaggtt

SEQ ID NO: 292 - DNA, 27 bp, synthetic construct
  source 1..27: mol_type = other DNA; note = hCD71_candidate#145
gcccaaactcctatgttggttcgtggt

SEQ ID NO: 293 - DNA, 27 bp, synthetic construct
  source 1..27: mol_type = other DNA; note = hCD71_candidate#146
gcccaaccgctgacggcggctcgtcgg

SEQ ID NO: 294 - DNA, 27 bp, synthetic construct
  source 1..27: mol_type = other DNA; note = hCD71_candidate#147
gcccaagtgtttcataagcttcgttcg

SEQ ID NO: 295-297 - skipped (3 entries)

SEQ ID NO: 298 - protein, 736 aa, synthetic construct
  source 1..736: note = capctrl
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKAAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFNYEFENVPFHSSYAHSQSLDRLMNPLIDQYLYYLSKTINDSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADEVMITNEEEIKTTNPVATESYGQVAINHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 299 - skipped

SEQ ID NO: 300 - protein, 9 aa, synthetic construct
  source 1..9: note = consensus sequence
  VARIANT 3..4: note = X may be any amino acid.
  VARIANT 7..9: note = X may be any amino acid.
AQXXRLXXX

SEQ ID NO: 301 - protein, 743 aa, synthetic construct
  source 1..743: note = consensus sequence capctrl
  VARIANT 589..595: note = X may be any amino acid.
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKAAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFNYEFENVPFHSSYAHSQSLDRLMNPLIDQYLYYLSKTINDSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADEVMITNEEEIKTTNPVATESYGQVAINHQSAQXXXXXXXAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 302 - protein, 736 aa, synthetic construct
  source 1..736: note = capctrl-wt
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 303 - protein, 736 aa, synthetic construct; identical to SEQ ID NO: 302
  source 1..736: note = capctrl453D

SEQ ID NO: 304 - protein, 743 aa, synthetic construct
  source 1..743: note = AAV QX-230
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFNYEFENVPFHSSYAHSQSLDRLMNPLIDQYLYYLSKTINDSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADEVMITNEEEIKTTNPVATESYGQVAINHQSAQMHRLQTQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 305 - protein, 743 aa, synthetic construct
  source 1..743: note = AAV QX-269
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINDSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQMHRLQTQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 306 - protein, 743 aa, synthetic construct
  source 1..743: note = AAV QX-270
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQMHRLQTQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 307 - protein, 743 aa, synthetic construct
  source 1..743: note = consensus sequence AAV9 wt
  VARIANT 589..595: note = X may be any amino acid.
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQXXXXXXXAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 308 - protein, 743 aa, synthetic construct; identical to SEQ ID NO: 307
  source 1..743: note = consensus sequence AAV9 453D
  VARIANT 589..595: note = X may be any amino acid.